(19) United States
(12) Patent Application Publication
(10) Pub. No.: US 2025/0265373 A1
(43) Pub. Date: Aug. 21, 2025

(54) ANALYZING DATA STORED IN SEGREGATED DATA ENVIRONMENTS

(71) Applicant: Privacy Analytics Inc., Ottawa (CA)

(72) Inventors: Lon Michel Luk Arbuckle, Ottawa (CA); Devyani Priyambada Biswal, Toronto (CA)

(21) Appl. No.: 19/054,065

(22) Filed: Feb. 14, 2025

Related U.S. Application Data
(60) Provisional application No. 63/740,168, filed on Dec. 30, 2024.
(60) Provisional application No. 63/554,651, filed on Feb. 16, 2024.

(51) Int. Cl. G06F 21/62; G16H 10/60

(52) U.S. Cl. CPC G06F 21/6254; G16H 10/60

(57) ABSTRACT

Methods and systems for generating a synthetic trends dataset by securely combining a healthcare dataset with a supplementary dataset, each pertaining to a common individual. A system receives the healthcare dataset and a supplementary dataset from databases in respective segregated data environments of a federated data cleanroom. The system anonymizes each dataset, stores the anonymized data in respective segregated data environments, and generates numerical representations of data features that include sequences of embedding vectors. The system transforms the sequences of embedding vectors by reducing the information with respect to the original sequences of embedding vectors. Upon determining a risk of disclosure is above a threshold, the system modifies the data features. The system generates a synthetic trends dataset that includes the transformed sequence of embeddings associated with each segregated data environment and outputs values from a machine learning model that is trained with the synthetic trends dataset.

### CLAIM OF PRIORITY

This application claims priority under 35 USC § 119 (e) to U.S. Patent Application Ser. No. 63/554,651, filed on Feb. 16, 2024, and U.S. Patent Application Ser. No. 63/740,168, filed on Dec. 30, 2024, the entire contents of which are hereby incorporated by reference.

### BACKGROUND

This specification relates to analyzing data from multiple segregated data environments.

Data are often segregated across different systems and organizations due to privacy laws, intellectual property concerns, and regulatory requirements. For instance, health data is subject to regulations such as the Health Insurance Portability and Accountability Act (HIPAA), which restricts the sharing of sensitive patient information without appropriate safeguards. Similarly, consumer data, including purchase histories and behavioral information, is governed by regulations such as the General Data Protection Regulation (GDPR) to protect individual privacy and consent. These restrictions provide challenges to combining different types of data in a shared data environment, even though such integration could provide valuable insights for a variety of applications.

### SUMMARY

The systems and techniques described here relate to processing, analyzing, and combining data stored in segregated data environments of a federated data cleanroom. A federated data cleanroom is a group of segregated data environments in which data is stored and processed, and in which there is no direct linking of individual person data between the environments.

In some cases, data stored in different data environments (e.g., different servers with distinct access protocols) cannot be combined due to privacy, security, intellectual property, and/or contractual restrictions. As such, linking data with different attributes stored in different data environments (as is desirable for certain data analytics and machine learning tasks) can increase a risk of identification of a data subject (e.g., an individual or a patient) based on the stored data. For example, there can be a risk of identifying the data subject from pseudonyms or tokens that are used to link data across datasets, especially in the case of linking health data to online identifiers for audience activation (e.g., online direct to consumer marketing).

The disclosed techniques of this specification allow for combining and analyzing data from multiple segregated data environments in a single data environment. Although the data from the segregated data environments cannot be directly combined, the disclosed techniques describe a method for transforming the segregated data to minimize a risk of disclosing individuals represented in the data. Before combining the data from the segregated environments, each dataset is transformed into a numerical representation (e.g., numerical vectors), which combines confidentiality protection and de-identification to prevent dataset reconstruction attacks and protect against emerging AIML threats, and processed with one or more privacy enhancing techniques (e.g., dimensionality reduction and noise injection). The transformation and application of privacy enhancing techniques allows for the combination of datasets from segregated data environments to provide high quality target audience groups. For example, the combined data fields can include data related to consumer activity alongside health data while mitigating a risk of disclosing associated individuals. The combined data can be used to train a machine learning model or to generate other analytical insights.

The subject matter described in this specification can be implemented in particular embodiments to realize one or more of the following advantages. Techniques are described for implementing a method for combining and analyzing data from multiple segregated data environments while minimizing a risk of disclosure of individuals that have representative data stored in one or more of the multiple data environments.

The application of privacy enhancing techniques provide an auditable and provable degree of privacy while enabling advanced analysis on a combined data set that includes data from multiple segregated data environments. Furthermore, the disclosed techniques of this specification allow for tuning the data transformations (e.g., modifying a feature selection procedure and/or specific privacy enhancing techniques) that occur before data combination to optimize a tradeoff between a degree of utility (e.g., usefulness of the combined dataset for addressing a particular task or question) and a risk of disclosure (e.g., a probability of an individual being identified based on the combined dataset). By pre-processing the data stored in the segregated data environments before combination, the disclosed techniques minimize a risk of identification and breaching restrictions (e.g., regulatory restrictions) of comingling the various data sources. In particular, the disclosed specification enables incorporation of health data into audience modeling for consumer marketing applications while minimizing a risk of disclosing personally identifiable information (PII). In addition to health data, the described methods are applicable to other industries including finance, defense, government, and others that require separation of confidential and/or private data.

The methods described in the present specification include a federated learning approach, in which data represented in a latent space (e.g., a lower dimensional vectorized embedded representation of the data) are shared between data environments instead of the data itself. Unlike conventional methods (e.g., sharing the raw data or de-identified data) that require centralized data aggregation, the present approach ensures that sensitive data remain siloed within a respective data cleanroom. A combination of principal component analysis (PCA), which is a dimensionality reduction approach, and federated orchestration transforms data into a shared embedding space for modeling, enabling cooperation between data environments without sacrificing security and/or ownership of underlying data. PCA and related techniques (e.g., techniques for reducing the dimensionality of a dataset) satisfy transparency and explainability requirements of responsible AI guidelines.

Furthermore, by automating data transformations, latent space generation, and privacy controls, the present approach results in a reduction of a reliance on manual interventions, thereby reducing errors due to manual intervention and improving scalability of associated systems. The PCA-based system can be integrated into data management systems (e.g., AI data management) that handle risk assessments, performance monitoring, and compliance with associated validations and minimal manual oversight.

The latent space associated with the shared data in each data environment reduces a required data storage for data sharing and machine learning modeling tasks. Only the data features (embedding vectors or linear combinations of embedding vectors) that exhibit the highest variance are retained. Low-variance data features are discarded. As a result, it is more efficient to manage large-scale datasets for machine learning tasks (e.g., training). Furthermore, by reducing the size of the data transferred between compute units, the present approach reduces processing times (e.g., time required to train a model) while enhancing security, as smaller and abstracted datasets are more difficult to exploit. Additionally, the data compression (e.g., representation in an embedding space and/or a reduced dimensionality embedding space), eliminates a need for extensive model training and/or knowledge transfer regarding classification of sensitive data attributes, as data is transformed uniformly across all data fields.

Representing data in a latent space reduces a risk of reconstruction attacks, in which an adversary has access to data and an attribute dictionary and is able to reconstruct an original dataset with sensitive information from the latent space representation of the dataset. The present approach provides flexibility to mitigate this risk by adjusting an amount of dimensionality reduction (e.g., retaining less variance), incorporating non-linear transformations and/or introducing randomized noise to further obscure data relationships, and to limit access to the attribute dictionary by only providing (and storing) aggregate-level information and/or simplified metadata in relation to the original data that may include sensitive information.

In one aspect, a method for generating a synthetic trends dataset by securely combining a healthcare dataset pertaining to an individual with a supplementary dataset pertaining to the individual includes receiving the healthcare dataset from a database in a first segregated data environment of a federated data cleanroom. The healthcare dataset including personally identifiable information (PII) pertaining to the individual. In addition, the method includes receiving the supplementary dataset from a database in a second segregated data environment of the federated data cleanroom, in which the supplementary data comprising PII pertaining to the individual. The method further includes anonymizing the data stored in each database, in which the anonymized data from each database is stored in the corresponding segregated database, generating, for each segregated data environment, a numerical representation of each data feature of multiple data features of the anonymized data stored in the corresponding segregated data environment, in which the numerical representation includes a first sequence of embedding vectors. The method includes determining, for each segregated data environment, a second sequence of embedding vectors. The second sequence of embedding vectors is a transformation of the corresponding first sequence of embedding vectors, the transformation including a reduction of information from the first sequence of embedding vectors. Furthermore, the method includes modifying, upon determining a risk of disclosure is above a disclosure threshold, the data features for a corresponding segregated data environment, in which the risk of disclosure is indicative of a likelihood that the PII of the data stored in a database of the corresponding segregated data environment is obtainable from the corresponding second sequence of embedding vectors. The method includes generating the synthetic trends dataset that includes the second sequence of embedding vectors associated with each segregated data environment, and outputting, from a machine learning model trained on the generated synthetic trends dataset, an output value that is indicative of a probability that the individual takes a particular action based on the health dataset and the supplementary dataset pertaining to the individual.

In some implementations, the transformation of the first sequence of embedding vectors includes reducing a dimensionality of the first sequence of embedding vectors. In some implementations, the transformation of the first sequence of embedding vectors includes a lossy compression of the first sequence of embedding vectors. In some implementations, the transformation of the first sequence of embedding vectors includes adding noise to the first sequence of embedding vectors, in which the noise includes one or more sources of noise.

In some implementations, the generation of the first sequence of embedding vectors for each segregated data environment includes a principal component analysis of the corresponding dataset.

In some implementations, the method further includes generating a token from the PII of each dataset pertaining to the individual, in which the token is operative to link the corresponding dataset to data stored outside of the federated data cleanroom.

In some implementations, the method further includes determining a utility of the dataset, in which the utility is indicative of a quality of the dataset with respect to a particular task, determining that the utility of the dataset is below a utility threshold that represents a minimum required quality of insights generated based on analytics of the dataset, modifying, based on the utility of the dataset being below the utility threshold, one or more of the determined data features to increase the utility of the dataset, and after the modifying, outputting insights generated based on analytics of the dataset.

In some implementations, determining the risk of disclosure includes determining a k-anonymity metric, in which the k-anonymity metric depends on a signal-to-noise ratio (SNR) and a similarity probability of each data point of the second sequence of embedding vectors.

In some implementations, generating the first sequence of embedding vectors includes capturing a variance of the anonymized data in fewer dimensions than the dimensionality of the anonymized data.

In some implementations, the healthcare data includes a multiple alphanumeric codes, in which each alphanumeric code is mapped to an embedding vector.

In another aspect, a system includes one or more computers and one or more computer-readable media storing instructions that are operable, when executed by the one or more computers, to perform operations for generating a synthetic trends dataset by securely combining a healthcare dataset pertaining to an individual with a supplementary dataset pertaining to the individual. The operations include generating a synthetic trends dataset by securely combining a healthcare dataset pertaining to an individual with a supplementary dataset pertaining to the individual includes receiving the healthcare dataset from a database in a first segregated data environment of a federated data cleanroom. The healthcare dataset including personally identifiable information (PII) pertaining to the individual. In addition, the operations include receiving the supplementary dataset from a database in a second segregated data environment of the federated data cleanroom, in which the supplementary data comprising PII pertaining to the individual. The operations further include anonymizing the data stored in each database, in which the anonymized data from each database is stored in the corresponding segregated database, generating, for each segregated data environment, a numerical representation of each data feature of multiple data features of the anonymized data stored in the corresponding segregated data environment, in which the numerical representation includes a first sequence of embedding vectors. The operations include determining, for each segregated data environment, a second sequence of embedding vectors. The second sequence of embedding vectors is a transformation of the corresponding first sequence of embedding vectors, the transformation including a reduction of information from the first sequence of embedding vectors. Furthermore, the operations include modifying, upon determining a risk of disclosure is above a disclosure threshold, the data features for a corresponding segregated data environment, in which the risk of disclosure is indicative of a likelihood that the PII of the data stored in a database of the corresponding segregated data environment is obtainable from the corresponding second sequence of embedding vectors. The operations include generating the synthetic trends dataset that includes the second sequence of embedding vectors associated with each segregated data environment, and outputting, from a machine learning model trained on the generated synthetic trends dataset, an output value that is indicative of a probability that the individual takes a particular action based on the health dataset and the supplementary dataset pertaining to the individual.

In some implementations, the transformation of the first sequence of embedding vectors includes one or more of reducing a dimensionality of the first sequence of embedding vectors, a lossy compression of the first sequence of embedding vectors, and adding noise to the first sequence of embedding vectors, in which the noise includes one or more sources of noise.

In some implementations, the generation of the first sequence of embedding vectors for each segregated data environment includes a principal component analysis of the corresponding dataset.

In some implementations, the operations further include generating a token from the PII of each dataset pertaining to the individual, in which the token is operative to link the corresponding dataset to data stored outside of the federated data cleanroom.

In some implementations, the operations further include determining a utility of the dataset, in which the utility is indicative of a quality of the dataset with respect to a particular task, determining that the utility of the dataset is below a utility threshold that represents a minimum required quality of insights generated based on analytics of the dataset, modifying, based on the utility of the dataset being below the utility threshold, one or more of the determined data features to increase the utility of the dataset, and after the modifying, outputting insights generated based on analytics of the dataset.

In some implementations, determining the risk of disclosure includes determining a k-anonymity metric, in which the k-anonymity metric depends on a signal-to-noise ratio (SNR) and a similarity probability of each data point of the second sequence of embedding vectors.

In some implementations, generating the first sequence of embedding vectors includes capturing a variance of the anonymized data in fewer dimensions than the dimensionality of the anonymized data.

In some implementations, the healthcare data includes a multiple alphanumeric codes, in which each alphanumeric code is mapped to an embedding vector.

In another aspect, a non-transitory computer-readable medium storing one or more instructions executable by a computer system to perform operations for generating a synthetic trends dataset by securely combining a healthcare dataset pertaining to an individual with a supplementary dataset pertaining to the individual include generating a synthetic trends dataset by securely combining a healthcare dataset pertaining to an individual with a supplementary dataset pertaining to the individual includes receiving the healthcare dataset from a database in a first segregated data environment of a federated data cleanroom. The healthcare dataset including personally identifiable information (PII) pertaining to the individual. In addition, the operations include receiving the supplementary dataset from a database in a second segregated data environment of the federated data cleanroom, in which the supplementary data comprising PII pertaining to the individual. The operations further include anonymizing the data stored in each database, in which the anonymized data from each database is stored in the corresponding segregated database, generating, for each segregated data environment, a numerical representation of each data feature of multiple data features of the anonymized data stored in the corresponding segregated data environment, in which the numerical representation includes a first sequence of embedding vectors. The operations include determining, for each segregated data environment, a second sequence of embedding vectors. The second sequence of embedding vectors is a transformation of the corresponding first sequence of embedding vectors, the transformation including a reduction of information from the first sequence of embedding vectors. Furthermore, the operations include modifying, upon determining a risk of disclosure is above a disclosure threshold, the data features for a corresponding segregated data environment, in which the risk of disclosure is indicative of a likelihood that the PII of the data stored in a database of the corresponding segregated data environment is obtainable from the corresponding second sequence of embedding vectors. The operations include generating the synthetic trends dataset that includes the second sequence of embedding vectors associated with each segregated data environment, and outputting, from a machine learning model trained on the generated synthetic trends dataset, an output value that is indicative of a probability that the individual takes a particular action based on the health dataset and the supplementary dataset pertaining to the individual.

In some implementations, the transformation of the first sequence of embedding vectors comprises one or more of reducing a dimensionality of the first sequence of embedding vectors, a lossy compression of the first sequence of embedding vectors, and adding noise to the first sequence of embedding vectors, wherein the noise comprises one or more sources of noise.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 illustrates an example system for accessing datasets that are stored in segregated data environments.

FIG. 2 illustrates an example system for accessing datasets that are stored in segregated data environments.

FIG. 3 illustrates an example system for improving a feature selection process based on a utility threshold.

FIG. 4 illustrates an example system for improving a feature selection process based on evaluating a risk of disclosure.

FIG. 5 illustrates an example procedure for combining data from more than one segregated data environment.

FIG. 6 illustrates an example data transformation.

FIG. 7 illustrates an example randomization of health data.

FIG. 8 illustrates an example dataset that includes randomized flag attributes, a minimized dataset, and sample health data.

FIG. 9 illustrates an example evaluation process of machine learning model outputs.

FIG. 10 illustrates an example process for generating synthetic health trends.

FIG. 11 is a flow diagram of an example process for generating a dataset by combining data stored in more than one segregated database.

FIG. 12 is a graphical representation of a threshold pre-processing approach.

FIG. 13 illustrates an example system 1300 for combining data from two segregated data environments.

FIG. 14 is an example system architecture.

FIG. 15 is an example system architecture.

Like reference numbers and designations in the various drawings indicate like elements.

### DETAILED DESCRIPTION

The systems and techniques described here relate to methods for processing, analyzing, and combining data from multiple segregated data environments of a federated data cleanroom. The federated data cleanroom includes multiple data environments, in which data stored in each environment may be prohibited to be comingled with other data due to various privacy, security, intellectual property, and contractual restrictions. In some cases, a combination of data from more than one segregated databases (e.g., health data databases and consumer data databases) provides an opportunity for accessing richer insights by processing combined datasets with machine learning models.

These methods include transforming the data stored in each segregated database as a pre-processing step before the data is combined in an insights environment that contains data to be processed by a single machine learning model or data analysis process. The transformation includes de-identifying each dataset, converting each dataset into a numerical representation in an embedding space (i.e., a sequence of embedding vectors), performing one or more privacy enhancement techniques (e.g., dimensionality reduction, lossy compression, noise injection, etc.), and harmonizing identification tokens and/or pseudonyms to enable data linking of data associated with particular individuals between the data from each database. After the pre-processing steps (i.e., de-identification, embedding, privacy enhancement, and harmonization), the data from each segregated database are combined and can be processed and analyzed as a single dataset.

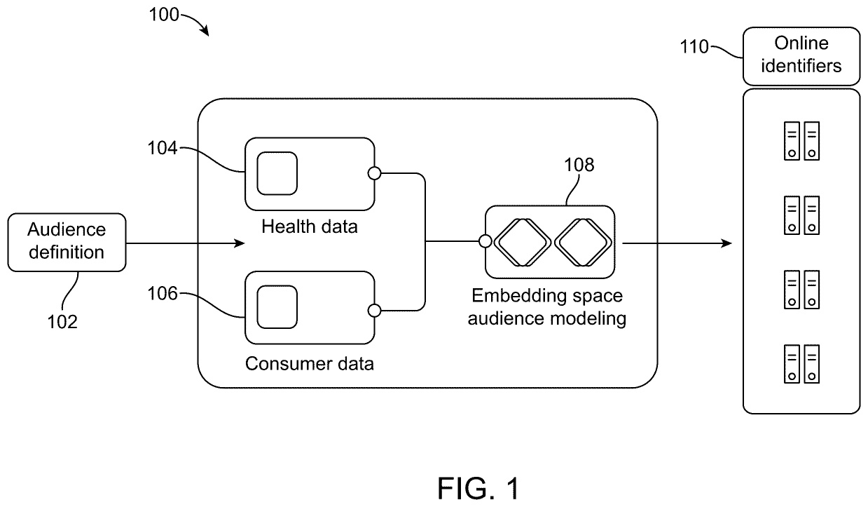

FIG. 1 illustrates an example system 100 for accessing and combining datasets that are stored in segregated data environments. The system receives an audience definition 102. In some implementations, the audience definition 102 includes parameters of a group of individuals (e.g., a patient cohort) that are associated with data stored in a health data database 104 and a consumer data database 106. For example, the audience definition 102 can include age ranges (e.g., patients aged 18 to 30), diagnoses (e.g., diagnosed with Type II diabetes), medication statuses (e.g., not currently on Metformin), and time frames (e.g., with a 5 year lookback).

In some cases, it is desirable to combine health data stored in the health data database 104 with data stored in the consumer data database 106. For example, data stored in the consumer data database 106 can include household income, marital status, education level, etc. Example cases include direct to consumer marketing to individuals with particular demographics (e.g., data found in the consumer data database 106) and particular health-related conditions (e.g., data found in the health data database 104). In some cases, a simple combination of healthcare data and consumer data breaches privacy regulations, intellectual property, and/or contractual limitations. As such, this disclosure describes an approach for combining data from the health data database 104 and the consumer data database 106 that retains privacy of individuals described by the audience definition 102 and allows for an analysis of health data alongside consumer data.

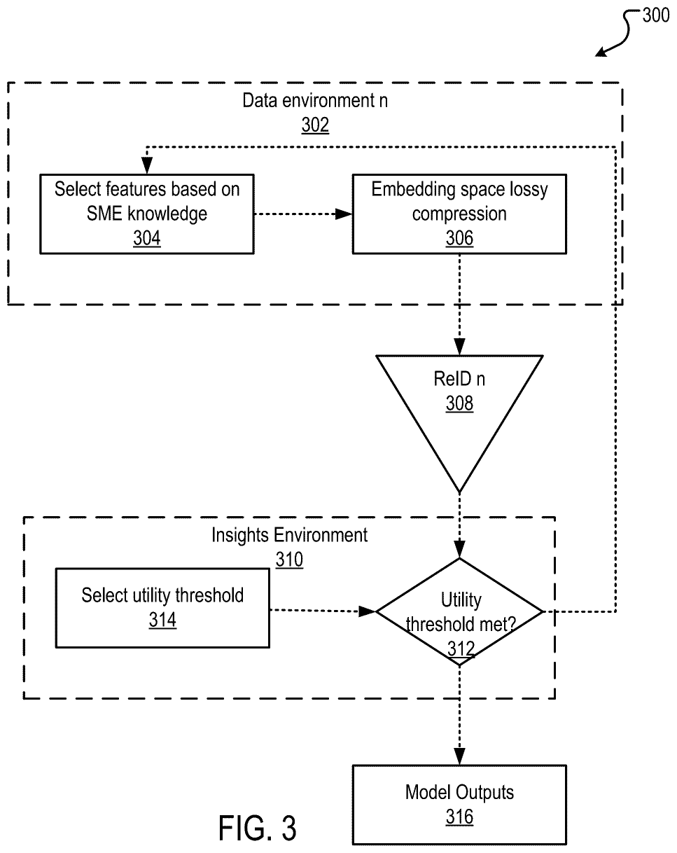

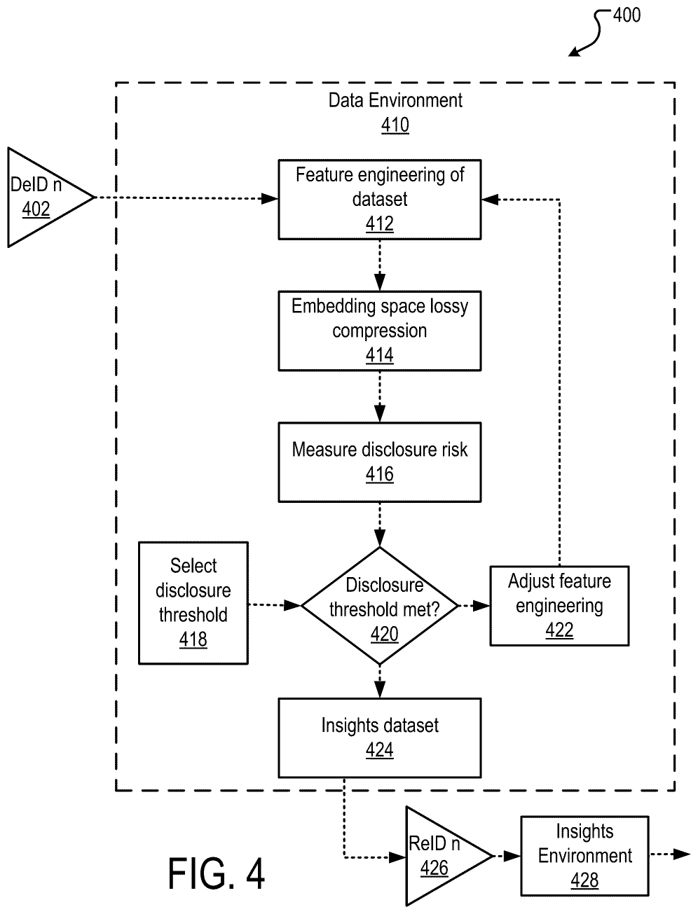

An embedding space audience modeling system 108, described in detail in the descriptions of FIGS. 3 and 4, processes data associated with individuals that are defined by the audience definition 102. The data is stored in the health data database 104 and the consumer data database 106. The system 108 generates an embedded representation of a subset of the health data and an embedded representation of a subset of the consumer data. An embedded representation is a numerical representation (e.g., a sequence of vectors) generated by a machine learning model that has been trained to convert data (e.g., words and phrases) into numbers that can be processed by a different machine learning model.

The system 108 transforms the embedded representations into a transformed embedded representation that include less information. In some implementations, the transformation includes adding noise to the embedded representations, selecting only the most important features of the embedded representation (e.g., via principal component analysis (PCA)), or performing a lossy compression of the embedded representations. Regardless of the chosen transformation method, the embedded representations undergo one or more privacy enhancing techniques (e.g., compression, noise injection, dimensionality reduction, etc.) that allows for combining data from multiple databases (e.g., the database 104 and the database 106) while reducing a risk of disclosing PII stored in either of the databases. The transformed embedded representations do not include enough information to reliably reconstruct data that links the contained insights to relevant PII of the individuals defined in the audience definition 102. Further detail describing various transformation operations are described in the figures below.

In some implementations, the system 108 includes a machine learning model training system that trains a machine learning model with the transformed embedded representations. For example, the system 108 can train a machine learning model to generate an output indicative of whether a particular individual is likely to purchase a particular product based on associated health data and consumer data. Outputs of the machine learning model can be linked to online identifiers 110 (e.g., via a tokenized identifier) to associate the outputs with online activity (e.g., website visits, social media activity, etc.). In some implementations, the online identifiers 110 are marketing identifiers.

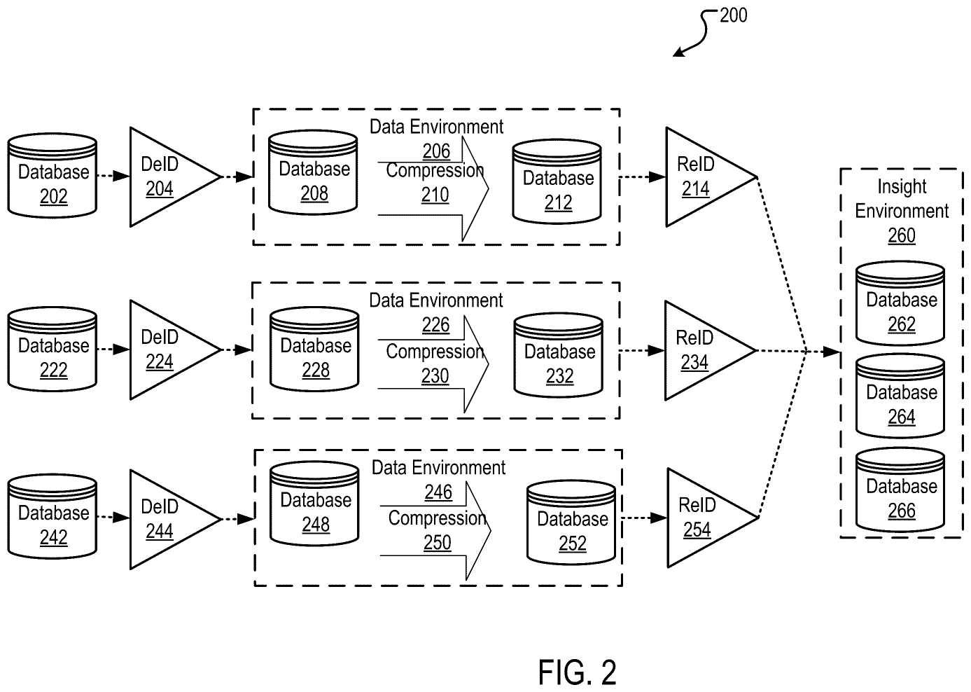

The system 100 facilitates a use of data stored in two segregated data environments (a health data environment and a consumer data environment) to generate an output based on both datasets. As described below in relation to FIG. 2, more than two segregated data environments can be combined to generate relevant insights related to particular individuals.

FIG. 2 illustrates an example system 200 for accessing and analyzing datasets that are stored in segregated data environments. The example system 200 illustrates three segregated data environments in the form of distinct databases 202, 222, and 242. For each database, a de-identification system 204, 224, 244 respectively removes all PII and PHI from the data associated with particular individuals. In some implementations, the particular individuals are defined by an audience definition (e.g., the audience definition 102).

The de-identification systems 204, 224, and 244 remove personal information from the data stored in the associated databases 202, 222, 242. For example, particular data fields are considered to be PII and are stored in the database 202. The fields can include patient name, email address, social security number, home address, and other data fields that can disclose the identity of a particular patient. In some implementations, the system 204 deletes PII from the data stored in the database 202. In some implementations, the system 204 masks or redacts PII from the data stored in the database 202. For example, a name “John Doe” can be replaced with “Patient123.” In some implementations, the system 204 groups PII fields into broad categories to reduce specificity. For example, the system 204 can replace precise age data stored in the database 202 with age ranges (e.g., 25-34). In some implementations, the system 204 replaces identifiers with pseudonyms or codes that cannot be directly traced back without a separate mapping. For example, the system 204 can replace names with random alphanumeric codes. In some implementations, the system 204 broadens the scope of fields that contain PII to reduce identifiability. For example, a data field that includes a patient's zip code can be replace with the first 3 digits of the zip code. In some implementations, the system 204 introduces noise into data fields that contain PII to mask the original information. For example, the system 204 can add a small random offset to income data, health metrics, or address geocoordinates.

After de-identification by the de-identification systems 204, 224, and 244, an associated data environment 206, 226, and 246 stores the de-identified data in respective de-identified databases 208, 228, and 248. In some cases, the data environments 206, 226, and 246 are segregated data environments in which the data stored in the databases 208, 228, and 248 cannot be comingled due to regulatory, intellectual property, and compliance reasons.

The data environments 206, 226, and 246 each include a respective compression module 210, 230, and 250. Each compression module transforms the data stored in the database 208, 228, and 248 into data stored in databases 212, 232, and 252. In some implementations, each of the compression modules 210, 230, and 250 implement one or more privacy enhancing techniques that reduce a probability of PII and PHI disclosure.

As an illustrative example, consider the compression module 210 and its associated data operations. Each of the compression modules (modules 210, 230, and 250) can implement one or more of the privacy enhancing techniques described below.

In some implementations, the compression module 210 can implement a feature selection process. In some cases, the feature selection process includes input from a subject matter expert, in which the expert determines one or more features (e.g., data fields, modified data fields, combinations of data fields, or combinations of modified data fields) that provide the most predictive value for a particular objective task. In some implementations, automated approaches to feature selection can be implemented (e.g., AutoML) in which features of the data are determined iteratively by evaluating utility/accuracy metrics of a machine learning model and modifying the selected features until a particular threshold is met.

In some implementations, the compression module 210 transforms the data stored in the database 208 into a numerical representation of data as a sequence of embedding vectors. A sequence of embedding vectors can capture underlying semantic, structural, and relational information present in the data stored in the database 208. Embedding vectors are often used in machine learning applications, particularly for processing unstructured data like text and images. In some implementations, the sequence of embedding vectors is represented as an embedding matrix, in which each row (or column) is interpreted as an embedding vector.

In some implementations, the compression module 210 implements one or more data pre-processing steps before generating the sequence of embedding vectors. For example, in some cases, the module 210 can normalize or clean the data (e.g., tokenize and lower-case words) and format the data to be processed by an embedding neural network model (e.g., convert words into token IDs, images into tensors, and categorical variables into indices).

In some implementations, the compression module 210 generates the sequence of embedding vectors using a mapping technique, in which each word (of text-based data) is mapped to a pre-defined vector. The set of vectors that are used to map each word can be generated by a trained embedding model (e.g., a trained neural network model), in which the trained embedding model processes the data stored in the database 208 (or potentially the data stored in the database 208 after a pre-processing step).

In some implementations, the compression module 210 transforms the generated sequence of embedding vectors with one or more privacy enhancing techniques. In some cases, the privacy enhancing techniques include reducing the dimensionality (e.g., fewer embedding vectors or smaller embedding vectors) of the generated sequence of embedding vectors. As such, the transformed sequence of embedding vectors contains less information than the generated sequence of embedding vectors while retaining enough structure for an objective analysis.

As an example of a privacy enhancing technique, the compression module 210 can perform a principal component analysis (PCA), in which each embedding vector is projected onto the directions of maximum variance in order to retain only the most important (e.g., vectors with the most variation) vectors. In some cases, PCA provides obfuscation to small details in the underlying data by removing less significant (e.g., vectors with little variation) dimensions.

The use of PCA as an approach to privacy protection represents a transformation of the entirety of a dataset, rather than selectively targeting identifiable attributes. The holistic transformation minimizes both attributional and inferential disclosure risks (risks of disclosing an identity of an individual), as the embedding space obfuscates all attributes of a data item including personal identifiers, sensitive information, confidential information, and correlated fields. Unlike traditional anonymization methods that rely on classifying identifiable and sensitive fields, PCA automatically aligns the data with principal components, eliminating the need for complex and often error-prone manual classifications of attributes. By ensuring that all data undergoes transformation (e.g., transformation into the embedding space), the approach inherently protects against threats like model inversion and membership inference, as the original data structure is not accessible.

The embedding space (also referred to as latent space or synthetic trends) generated by the PCA process is uninterpretable, which adds a layer of defense against adversarial attacks. The embedding space retains trends and patterns in the data, but the data are abstracted into linear combinations of the principal components. In addition, the obfuscation generated through the PCA decomposition process can be complemented by an injection of noise/randomization (i.e., differential privacy), which introduces a controlled noise to further secure the embedding space against AI-driven (e.g., pattern recognition) threats. The combination of dimensionality reduction (PCA decomposition) and differential privacy provides a safeguard that mitigates risk associated with dataset reconstruction attacks, re-identification, attribution, and inferential disclosure risks (confidentiality) of data fields in federated data environments.

PCA-based security enhancement offers data security (e.g., it is difficult to reconstruct the original data) and data utility (the trends and patterns of the data are retained, since the majority of variation in the data are along the principal axes). The embedding space is more robust for training machine learning models. Irrelevant attributes are removed, and computational complexity is reduced.

In some implementations, the compression module 210 performs a longitudinal PCA approach to address longitudinal relationships (e.g., relationships between data items for a particular patient over time). The longitudinal PCA approach includes a functional PCA (FPCA) and a multi-way PCA (MPCA).

FPCA is an extension of PCA that deals with functional data, which can include longitudinal data. Instead of treating each observation (e.g., data entry) independently, FPCA treats the data as smooth curves over time, making the approach suitable for capturing temporal dependencies. FPCA is designed to analyze and decompose data that evolves over time, ensuring that extracted health insights encapsulate critical temporal patterns.

MPCA extends PCA to handle multi-dimensional data, including longitudinal data by treating time as an additional mode. MPCA reduces the dimensionality of data across all modes, including time, while retaining interdependencies between modes.

In addition to PCA, alternative dimensionality reduction techniques can be employed including linear methods (e.g., singular value decomposition (SVD) and factor analysis) and nonlinear methods (e.g., t-distributed stochastic neighbor embedding (t-SNE), uniform manifold approximation and projection (UMAP), and autoencoders). Each technique transforms high-dimensional data into a compressed latent space, abstracting direct and indirect identifiers while preserving critical patterns and relationships for machine learning models.

In a general sense, the compression module 210 can implement techniques of a broad class of embedding space construction techniques in place of PCA and its variants. Embedding space construction maps a dataset onto an embedding space/latent space. In some implementations, a system processes (e.g., with a machine learning model) on the data represented in the embedding space/latent space instead of the original dataset. The specific approaches (e.g., PCA, FPCA, MPCA, etc.) discussed above are specific implementations of embedding space construction.

As another example of a privacy enhancing technique, the compression module 210 can process the sequence of embedding vectors with an autoencoder as a lossy compression of the data. An autoencoder represents the sequence of embedding vectors in a lower dimensional space, similar to PCA. The autoencoder is a layer of a neural network that can be learned and tuned to optimize for a particular performance metric.

As another example of a privacy enhancing technique, the compression module 210 can introduce random noise to the sequence of embedding vectors using a single source or noise or multiple sources of noise simultaneously.

The transformed sequence of embedding vectors after being processed with a privacy enhancing technique are stored in the database 212. Similarly, the transformed sequences of embedding vectors associated with the data environments 226 and 246 are stored in the databases 232 and 252 respectively. In some cases, the privacy enhancing techniques implemented by the compression modules 210, 230, and 250 are the same. In some other cases, the compression modules 210, 230, and 250 implement unique privacy enhancing techniques appropriate for the particular type of data stored in the respective databases 208, 228, and 248.

For each database 212, 232, and 252, an associated re-identification system 214, 234, and 254 respectively process the data by harmonizing one or more data fields in the data stored in the databases 212, 232, and 252. In some implementations, the data include pseudonyms or tokens introduced by the de-identification systems 204, 224, and 244, depending on the particular de-identification approach implemented by each system 204, 224, and 244. The re-identification systems 214, 243, and 254 modify one or more fields that are used to link data pertaining to an anonymized individual (e.g., a patient) with data pertaining to the same individual stored in one of the other anonymized and transformed databases. For example, the re-identification system 214 can modify pseudonyms in the data stored in the database 212 to match particular pseudonyms present in the data stored in the database 252 in order to link the two datasets together.

An insight environment 260 receives or accesses the data stored in the databases 212, 232, and 252 after the data is processed by associated re-identification systems 214, 234, and 254 respectively. In some implementations, the insight environment 260 includes databases 262-266 for storing the re-identified data associated with each data source. The data stored in the databases 262-266 represents a version of the data stored in the databases 202, 222, and 242 after de-identification, embedding, compression, and re-identification. The aforementioned processes allow for a scenario in which the data stored in the databases 262-266 can be comingled and analyzed together in the insights environment 260. In some implementations, the data stored in the databases 262-266 are used for training a machine learning model.

FIG. 3 illustrates an example system 300 for improving a feature selection process based on a utility threshold. The system 300 includes an example data environment 302 and an example insights environment 310. The example environments 302 and 310 are analogous to the environments 206 and 260 described in relation to FIG. 2. The present description of the example system 300 provides additional detail into the compression module 210 of FIG. 2 and how the feature selection process is modified based on evaluating the utility of a machine learning model trained based on a transformed sequence of embedding vectors generated by the data environment 302 (i.e., the data environment 206).

As illustrated in relation to the data environment 206 of FIG. 2, the data environment 302 receives de-identified data associated with a particular data source. In some implementations, the data source is related to health data of a patient. In some other implementations, the data source is related to consumer data (e.g., online activity) of an individual.

Operations executed by modules (software programs) within the data environment 302 (e.g., compression module) are associated with multiple data processing steps. The data processing steps can be implemented on one or more computers, processors, or servers. The data environment 302 includes executable instructions for implementing a feature selection procedure 304. Feature selection is a process of identifying features in a dataset to optimize outputs of machine learning models, reduce computational complexity, and/or to prevent overfitting of data by a machine learning model. Various approaches to feature selection are possible.

In some implementations, the feature selection procedure 304 includes an implementation of statistical measures to evaluate relevance of features (e.g., data fields or combinations of data fields) independent of any machine learning model. For example, the procedure 304 can include a correlation analysis in which a linear relationship between features and target variables is determined. The correlation analysis helps identify highly correlated features and variables. For example, if a machine learning model is configured to predict a value of x and a dataset has a variable y that is highly correlated with x, the correlation analysis identifies y as a feature of the dataset with predictive value. The procedure 304 can include other related statistical analyses like a measure of mutual information (a measure of information one feature provides about a target variable) and an application of a variance threshold (a removal of features with low variation).

In some implementations, the feature selection procedure 304 includes application of subject matter expertise in the selection of relevant features in a dataset. In some cases, subject matter expertise can be stored in a database or file and applied as a starting point for automated feature selection protocols.

In some implementations, the feature selection procedure 304 includes an iterative determination and adjusting of features by training a machine learning model on a set of selected features, validating outputs of the model, and iteratively determining a set of features that perform the best. In some implementations, the procedure 304 includes one or more embedded feature selection methods in which feature selection is implemented as part of the model training process which includes tree-based methods. In some implementations, the procedure 304 includes a combination of multiple methods of feature selection, also referred to as feature engineering. The particular approach (e.g., which features are chosen and how they are chosen) of can be modified based on one or more feedback mechanisms of the example system 300.

In addition to the feature selection procedure 304, the data environment 302 includes executable instructions for implementing an embedding procedure with lossy compression 306. As described in relation to FIG. 2, an embedding procedure includes converting data (in this case, the features determined by the feature selection procedure 304) into a numerical representation that consists of sequence of embedding vectors. The embedding procedure with lossy compression 306 includes data processing steps of (i) converting the selected features into a sequence of embedding vectors and (ii) implementing a lossy compression (e.g., reducing the dimensionality of the sequence of embedding vectors) to the sequence of embedding vectors. The lossy compression data processing step is one example of a privacy enhancing technique as described in relation to FIG. 2.

A re-identification system 308 harmonizes identification tokens or pseudonyms of the compressed sequence of embedding vectors to coincide with other data environments that may be associated with other data sources (e.g., other health data or consumer data related to a common individual). The harmonization is required to link data to particular individuals when the data originates from various different data sources with potentially different de-identification processes and formats.

An insights environment 310 receives the re-identified sequence of embedding vectors from the re-identification system 308 and processes the vectors with a trained machine learning model. The trained machine learning model generates a model output along with an evaluation of a utility of the model output. The utility of the model output is indicative of how useful the model output is for a particular task or objective. For example, the model output can be compared with a set of ground truth values in a set of training data to determine how accurate the machine learning model is.

The insight environment 310 includes a mechanism for selecting a utility threshold 314. In some implementations, the mechanism for selecting a utility threshold 314 includes reading a data file or database entry that represents the utility threshold. In some other implementations, the mechanism for selecting the utility threshold 314 includes receiving the utility threshold from a user interface. The insights environment 310 implements a utility threshold comparison 312 to determine if the model output of the machine learning model meets the selected utility threshold based on processing the received re-identified sequence of embedding vectors. If the utility threshold is not met, an instruction is received by the data environment 302 from the insights environment 310 (e.g., through a communication channel like an application programming interface (API) or a database flag) to modify the feature selection procedure 304. If the utility threshold is met, model outputs 316 of the machine learning model can be used in downstream applications.

In some implementations, based on the utility threshold not being met, the feature selection procedure 304 can modify the selected features and/or modify the procedure for selecting the features. In addition to modifying the feature selection procedure 304 based on the utility threshold not being met, the feature selection procedure 304 can also be modified based on other thresholds, including a risk of disclosure threshold, as described below in relation to FIG. 4.

FIG. 4 illustrates an example system 400 for improving a feature selection process based on evaluating a risk of disclosure. Like the example system 400, the example system 400 includes a feature selection procedure 412 (i.e., the feature selection procedure 304) and an embedding procedure with lossy compression 414 (i.e., the embedding procedure with lossy compression 306). The description of these two procedures in relation to FIG. 4 is the same as the description provided in relation to FIG. 3. The data environment 410 receives de-identified data from a de-identification system 402, as described in previous figures, and processes the received de-identified data with the corresponding feature selection procedure 412 and the embedding procedure with lossy compression 414.

The data environment 410 includes a disclosure risk evaluation 416. The disclosure risk evaluation 416 outputs a risk of disclosure metric that is indicative of how likely it is for an individual to be identified or inferred by the data after de-identification, embedding, and compression and other privacy enhancing techniques (e.g., dimensional reduction, noise injection, etc.). Risks of identify disclosure are present due to AI security threats such as model inversion (e.g., inverting an embedding model) and membership inference (e.g., identifying or inferring an individual based on the individuals membership to a group via a group characteristic).

Disclosure risks include risks or reconstruction (e.g., reconstructing a data set from an embedded representation of the dataset) and risks of attribute inference (e.g., inferring which attributes are present in a dataset through an analysis of the embedded representation of the dataset).

Reconstruction risks via reconstruction attacks on an embedding space can result from an adversary with access to (i) auxiliary data and (ii) an attribute dictionary. As such, the adversary can reconstruct an original dataset from the embedded representation of the dataset. To mitigate this risk, a system can apply strict dimensionality reduction to retain less variance in the dataset (e.g., retain fewer vectors during PCA), thereby reducing a potential for reconstruction.

Additionally, a system can incorporate nonlinear transformations or introduce randomized noise in an embedded representation to further obscure the relationship between the original data and the embedded representation. Furthermore, a system can limit access to the attribute dictionary by providing only aggregate-level information or simplified metadata related to the original dataset.

In addition to reconstruction attacks, a system can experience reconstruction risk due to high-variance retention (i.e., retaining high-variance vector components that preserve identifiable patterns, which can reveal sensitive information present in the original dataset). To mitigate this risk, a system can set a conservative variance retention threshold (e.g., 50%-70%) to balance data utility and privacy. Additionally, a system can employ regularized embedding generation and/or apply sparsity constraints to prevent any single component from dominating (e.g., most of the variance present in a dataset represented by a single vector component). Furthermore, a system can conduct risk assessments on retained components to identify and mask potential sensitive patterns.

In addition to risk of reconstructing the original dataset from the embedded representation, a dataset can be at risk of an adversary inferring the data attributes present in the original dataset through determining linkability through patterns. Patterns in the embedding space may correlate with specific sensitive attributes (e.g., social security number), which enables indirect identification of the sensitive attributes. To mitigate this risk, a system can perform an embedded correlation analysis between latent components (e.g., embedding vectors) and sensitive attributes of the original dataset to detect and mitigate correlations (i.e., risks). In addition, a system can mask and/or remove components of the embedding space that are highly correlated to identifiable features of the original dataset before sharing the embedding space with another party. Furthermore, a system can regularly test for inferential linkages using adversarial simulations to identify potential vulnerabilities.

In addition to an attribute inference risk due to linkability through patterns, an attribute inference risk can also be due to overlapping datasets. In other words, combining embedding space data with external datasets can enable statistical matching or inference of sensitive attributes. To mitigate this risk, a system can restrict sharing of the embedding space to trusted parties and establish strict data usage agreements that prohibit data linking between datasets. In addition, a system can monitor for misuse of shared data (e.g., shared embedded representations) by auditing outputs and tracking model performance (e.g., a machine learning model that processes the embedded representation) for potential linkages. Furthermore, a system can implement differential privacy mechanisms during embedding space generation to limit the impact of auxiliary data on generating attribute inference risks.

Risks of disclosure due to attribute dictionaries and analysis of model outputs can result in misuse of the data (e.g., healthcare data and/or consumer data with PII). To mitigate risks from attribute dictionaries and model outputs, a system can apply a cohort size threshold and noise injection to obfuscate patterns between datasets and to reduce the potential for linking data attributes to particular individuals.

In particular, a risk due to the presence of an attribute dictionary associated with a dataset is an auxiliary knowledge exploitation risk, in which access to the attribute dictionary can allow recipients of an embedded dataset to estimate feature contributions, which can narrow possible data values present in the original dataset. To mitigate auxiliary knowledge exploitation risk, a system can provide only high-level descriptions of attributes or general categories instead of a full attribute dictionary. In addition, a system can use controlled vocabularies with reduced granularity to minimize the specificity of attributes available to the recipient. Furthermore, a system can conduct scenario-based risk assessments to evaluate the potential misuse of auxiliary knowledge.

In addition to the auxiliary knowledge exploitation risk, a risk of statistical prior knowledge results from a recipient familiar with attribute distributions of a dataset mapping latent variables back to attributes of the dataset with high confidence. To mitigate this risk, a system can randomize feature contributions to ensure latent variables do not directly correspond to known attribute distributions. In addition, a system can add obfuscation layers, such as random projections, to reduce the interpretability of latent components.

Risks that originate from an analysis of model outputs result from tiered probabilities that may reveal trends that are correlated with sensitive attributes of a dataset if they are not properly aggregated. To mitigate this risk, a system can limit the resolution of tiered probabilities as an output from a model by expanding percentile ranges (e.g., 10th-90th percentiles). In addition, a system can regularly validate model outputs (e.g., audience model outputs) to ensure that the models to not align with sensitive data patterns in the dataset.

In addition to the risk from tiered probabilities, a risk of identifiability in outputs can result in a revelation of information about small, unique population subsets of a dataset. To mitigate this risk, a system can apply cohort size thresholds to ensure all modeled groups of individuals meet a minimum population size. In addition, a system can test outputs of a model for diversity and generality to ensure the outputs cannot be mapped to specific individuals or small groups.

Risk of disclosure due to implementation quality can be mitigated by techniques including nonlinear transformations, noise injection, and regularly updated threat models and testing strategies. In particular, implementation quality can result in risks due to poor embedding space configuration (e.g., weak orthogonalization, improper component selection, and suboptimal variance retention) that can lead to identifiable patterns to be detected in an embedded representation. To mitigate this risk, a system can use robust embedding space configuration tools that test and recommend optimal parameters for privacy and data utility. In addition, a system can regularly validate an embedding space configuration using privacy risk models to ensure no sensitive patterns are preserved during the transformation from a dataset to an embedded representation of the dataset. Furthermore, a system can train practitioners and implement quality assurance steps to avoid errors during embedding space setup.

In addition to implementation quality, risk of disclosure due to incomplete logging and documentation from weak logging practices can lead to errors and/or data leaks during data transformation, sharing, and usage. A system can mitigate this risk by implementing centralized logging for all embedding space processes, documenting transformation, decisions, and data flow. In addition, a system can conduct routing of internal audits of logs to ensure accuracy and to identify vulnerabilities. Furthermore, a system can use automated monitoring systems to detect anomalies and to provide alerts for any unexpected access or transformations.

In addition to risk or disclosure due to implementation risk, risks due to theoretical and practical limitations of a system can result from linear transformations and dependence on threat model assumptions. In particular, the linear nature of embedding space generation may miss complex non-linear relationships, which leaves residual identifiable patterns. To mitigate this risk, a system can implement nonlinear transformations, such as variational autoencoders, to capture and obscure nonlinear dependencies. In addition, a system can apply post-embedding space randomization or noise injection to reduce the interpretability of linear relationships. Furthermore, a system can test the embedding space with adversarial dataset reconstruction tools, including breaching confidentiality protections through re-identification, attribution, or inferential disclosure methods, to evaluate potential nonlinear vulnerabilities.

Risks due to a dependence on threat model assumptions occur when an adversary with access to auxiliary data might breach assumptions about the security of the latent space generated during embedding space generation. To mitigate this risk, a system can continuously update threat models to reflect evolving adversarial capabilities and emerging privacy risks. In addition, a system can conduct adversary testing using synthetic adversaries to assess the robustness of de-identification methods. Furthermore, a system can use ensemble de-identification methods that combine other privacy-preserving techniques (e.g., linear sensitivity rules and differential privacy).

The disclosure risk (i.e., re-identification, attribution, and inferential risks) evaluation 416 evaluates the risk of disclosure due to one or more of the described risks above. In some implementations, to perform the evaluation, a system translates an embedding space into measurable quantities. For example, a signal-to-noise ratio (SNR) is a measure indicative of an amount of meaningful signal (e.g., variance captured by the PCA procedure, as described in relation to FIG. 2) relative to an amount of noise introduced during a privacy-enhancing procedure. To ensure privacy (i.e., minimize risk of disclosure), high SNR data values are penalized. To minimize data points with high SNR, the contribution of high-SNR data points is decreased by applying an exponential function to each data point that decreases as SNR increases. The function can be written as

$$f ( SNR ) = {e}^{- \frac{SNR}{ϵ}} ,$$

where ϵ is an adjustable parameter that determines the sensitivity of the function to changes in SNR. In systems that utilize differential privacy, e represents a privacy budget that quantifies an allowable privacy loss. In other words, a smaller e results in stronger privacy. In some implementations, e depends on parameters related to noise injection into the dataset (e.g., statistical disclosure control, linear sensitivity rules, or differential privacy).

In addition to applying a SNR mitigating function, a system can evaluate a probability of similarity. The probability of similarity is indicative of how likely a data point in a latent space (embedding space) has “neighbors” within a specified range of a noise distribution. The probability of similarity considers clustering behavior of datasets and ensures k-noise reflects local density in the latent space. The probability of similarity can be expressed as,

$$P ( similarity ) = {\int }_{- δ}^{δ} {e}^{- \frac{{x}^{2}}{2 {σ}^{2}}} dx$$

where σ² represents a noise variance (reconstruction error), and δ represents a similarity threshold that defines the “neighborhood” around a datapoint in the latent space. The δ parameter determines the extent to which points around a data point are considered to be similar. Smaller threshold values increase privacy by requiring a tighter cluster, while larger threshold values improve utility by grouping more data points in the latent space together.

In some implementations, the threshold value (δ) can be expressed as a function of the noise variance by the expression, δ=cσ, where c is a scalar (e.g., 1 to 3) that determines a breadth of similarity based on a standard deviation of the noise. Relating the threshold value to the noise variation ensures that the probability adapts to an intrinsic variability of the noise introduced by PCA, resulting in the probability of similarity being grounded in measurable data properties. In other words, as the amount of noise is increased in a dataset, the “neighborhood” in which two datapoints are considered to be similar should be expanded as well.

To ensure privacy of a dataset, a k-anonymity metric can be evaluated as a combination of the exponential function of SNR and the similarity probability expressed above. The k-anonymity metric can be represented by assuming each embedding space dimension (e.g., principal component determined during PCA) is a quasi-identifier, where

$$k = \frac{f ( SNR )}{P ( similarity )} ,$$

where f (SNR) penalizes data points based on their respective SNR and P (similarity) that reflect local density in the latent space. The evaluation of f (SNR) addresses the uniqueness of data points, ensuring high-SNR points (distinct signals) contribute less to k-anonymity. Similarity probability ensures that the local density of the latent space reflects a likelihood of re-identification. Because both f (SNR) and P (similarity) depend on ø (reconstruction error or noise in the data), the k-anonymity metric ties privacy guarantees to the noise introduced by PCA.

In some cases, a system can use the k-anonymity metric to evaluate a disclosure risk. If a dataset has a low k-value (e.g., k=1 or k=2), some individuals can be uniquely identified or are in very small groups. Higher k-values (e.g., k=10 or k=50) reduce the risk of disclosure because each individual is indistinguishable from at least k−1 others. Thus, a dataset above a particular k-threshold is more robust against a linkage attack (where an attacker uses a quasi-identifier like age or zip-code to identify an induvial).

In some implementations, a system requires a determination of parameters that define the SNR function and probability of similarity, and thus the k-anonymity metric. The probability of similarity is indicative of how likely a data point in the latent space has “neighbors” within a specified range of the noise distribution, as determined by the PCA process. This probability accounts for clustering behavior and ensures that k-noise reflects local density in the latent space. A determination of ϵ is related to a privacy guarantee and is adjusted based on sensitivity of the data (e.g., based on regulatory benchmarks or empirical results). The size of the “neighborhood,” as defined by δ is related to the noise level (σ) and adjusted for neighborhood size, which can be adjusted to balance privacy of the dataset and utility of the dataset.

In some implementations, a system can adjust a risk measurement by factoring in noise that is introduced during a process of determining the embedded representation. When a system constructs an embedding space with noise injection (a privacy enhancing technique, e.g., statistical disclosure control or differential privacy), a system can introduce noise into the covariance matrix and/or norm/vectors during the computation of the embedding space vectors (e.g., eigenvalues and eigenvectors in relation to PCA). The noise obscures any precise relationships between original attributes of a dataset and vector representations in the embedding space (e.g., eigenvectors of PCA), which makes it harder for an adversary to reverse-engineer the original dataset. For example, in the case of differential privacy, the privacy budget e described above defines how much information is “leaked” per query of the original dataset. As more principal components are retained during PCA, an effective privacy leakage increases because (i) noise is distributed across more principal components, which reduces the individual sensitivity of each principal component and (ii) information content of higher-order principal components (those that explain less variance of the dataset) dimmish naturally, further reducing their utility for reverse-engineering.

Furthermore, as noise accumulates in the covariance matrix and/or embedding space vectors, (i) each embedding space dimension inherits a portion of the noise, and (ii) higher order dimensions, which explain less variance, are dominated by noise, which makes them less useful for adversaries that attempt reconstruction. Because higher-order embedding space dimensions contribute less to the overall explained variance of the dataset, (i) adversaries that attempt to infer the original data would require reconstructing lower-variance principal components, which are less representative of the dataset, and (ii) combined with noise, the reconstruction is increasingly infeasible as the number of retained embedding space dimensions (k) increases. In addition, with more retained embedding space dimensions, the number of possible combinations of original data attributes that could generate the dimensions grows exponentially. In other words (like entropy), an increase in the number of combinations reduces the likelihood of any single configuration being correct.

In some implementations, to model the decreasing identifiability as more embedding space dimensions are included, a system can implement an exponential decay factor based on the number of retained dimensions in the embedding space (d). In other words, a combination of noise accumulation in the covariance matrix and/or embedding vectors and the diminishing contribution of higher-order dimensions creates a compounding reduction of identifiability. An expression for an adjusted identifiability in view of these two effects can be expressed as

$$Adjusted Identifiability = \frac{{e}^{- \frac{SNR}{ϵ}} {e}^{- β d}}{n} ,$$

where the first exponential penalizes based on SNR, and the second exponential models the decreasing contribution of each additional embedding space dimension (e.g., principal component of PCA), 1/n represents a dilution effect of multiple contributing attributes to each embedding space dimension, and β is a tunable parameter that determines the rate of decay that reflects the impact of noise and diminishing variance in higher-order embedding space dimensions.

In some implementations, one or more parameters of the previously described expressions are determined based on one or more criteria. For example, the exponential decay rate β determines how quickly identifiability decreases as the number of embedding space dimensions increases. The value of β should be set to a value proportional to the privacy budget E (the effectiveness of noise injection depends on €). The privacy budget (E) governs the amount of noise added during embedding space construction and directly influences identifiability.

A combined equation that represents an embedding space equivalent to klatent can be written as

$${k}_{latent} = \frac{\frac{{e}^{- \frac{SNR}{ϵ}} {e}^{- \frac{β}{d}}}{P ( similarity )}}{n} .$$

The data environment 410 includes a mechanism for selecting a disclosure threshold 418. In some implementations, the mechanism for selecting a disclosure threshold 418 includes reading a data file or database entry that represents the disclosure threshold. In some other implementations, the mechanism for selecting the disclosure threshold 418 includes receiving the disclosure threshold from a user interface. The data environment 410 implements a disclosure threshold comparison 420 to determine if measured risk of disclosure by the disclosure risk evaluation 416 meets the selected disclosure threshold. If the disclosure threshold is not met (i.e., the risk of disclosure is too high), an instruction 422 is received by the system that implements the feature selection procedure 412 (e.g., through a communication channel like an application programming interface (API) or a database flag) to modify the feature selection procedure 412 to reduce the risk of disclosure. If the disclosure threshold is met, an insights dataset 424 can be generated and used for downstream applications in an insights environment 428. In some implementations, a re-identification system 426 harmonizes one or more identification tokens and/or pseudonyms between multiple data environments before the insights dataset 424 is processed and stored by the insights environment 428.

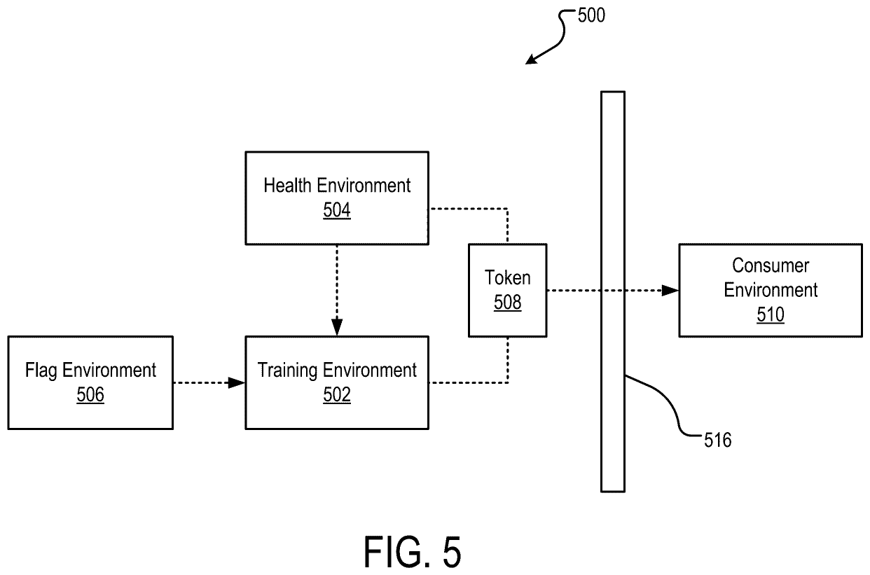

FIG. 5 illustrates an example data processing procedure 500 for combining data from more than one segregated data environment. The example procedure 500 includes processing data from a health environment 504, a flag environment 506, a training environment 502, and a consumer environment 510. A governance separation 516 that can generate tokens according to specific criteria for each side of the separation 516 separates the consumer environment 510 from the environments 502-506. In some implementations, the governance separation 516 is implemented by a third party that is not associated with the consumer environment 510 or the health environment 504.

The health environment 504 includes storage and processing of patient medical attributes. In some implementations, the patient medical attributes are specific to a particular company or organization. Possible organization-specific data assets include (i) patient-centric dataset that integrates company-specific pharmacy, lifecycle, and medical claims, (ii) ambulatory electronic medical records (AEMR), which is a patient-centric dataset that covers outpatient information about patient medical encounters (United States patients) including allergy, medication, procedures, diagnoses, orders, and results, and (iii) oncology electronic medical records (Oncology EMR), which includes data about cancer patients that have been active within the most recent five year period.

The health environment 504 includes systems (e.g., processors, computers, servers, etc.) for executing instructions related to dimensionality reduction (lossy compression) to the company-specific patient medical attributes to transform variables into a reduced dimension set of latent attributes (e.g., embedding vectors). In some cases, the reduced dimension set of latent attributes is referred to as minimized data. In some implementations, the system appends a crosswalk file to the minimized data and any company-specific patient identifiers (patient IDs) are removed and a token identified (token ID) is appended.

The health environment 504 outputs the minimized data with noisy and reduced statistics to the training environment 502. In some implementations, additional privacy measures can be added to the noisy minimized data by implementing differential privacy, which adds additional noise to the entries of the minimized data, rather than to the original data (the company-specific patient medical attributes and/or the associated embedded/numerical representations).

In some implementations, privacy enhancing techniques like dimensionality reduction results in medical attributes of the company-specific patient medical attributes being removed from any downstream applications. In some implementations, a particular percentage of variance in the features of the dataset (e.g., 95%) is allowed to pass to downstream applications to ensure a particular degree of loss, and as such, a particular degree of obfuscation of potential personal information.

In addition to receiving data from the health environment 504, the training environment 502 receives data from the flag environment 506. The flag environment 506 represents an environment that receives and processes model requests. A model request is a request for a machine learning model to be trained based on particular training data. In some implementations, the model requests are received through a user interface or an automated process (e.g., through an API call). The model request is indicative of an audience definition (e.g., a clinical audience type or a patient cohort). Each model request determines particular outcome variables to be generated by a trained machine learning model. In some implementations, the flag environment 506 includes the model request (e.g., a request to predict a particular output based on data stored in more than one segregated database), identifiers that are determined by sampling data from a first database (e.g., longitudinal prescription data (LRx)) and a second database (e.g., diagnosis data (Dx)) based on criteria defined by the model request, and a crosswalk file that maps identifiers between more than one segregated database.

The model request is received by the flag environment 506. The flag environment 506 executes instructions for defining a target patient cohort by applying criteria to the first database attributes and the second database attributes and receiving data indicative of individual identifiers from the first and second databases. The flag environment 506 generates a flag data attribute that is indicative of whether a particular individual represented by an individual identifier meets the criteria. In some implementations, the flag data attribute is set to 1 if the individual meets the criteria and it is set to 0 if the individual does not meet the criteria. In some implementations, after each individual identifier is assigned a flag data attribute based on whether the corresponding individual meets the criteria, one or more noise distributions are applied to the flag data attributes as a privacy enhancing technique. As such, some flag data attributes are changed to 1 that were previously 0 and vice versa to introduce uncertainty into the target patient cohort.

The flag environment 506 outputs a list of individual identifiers (e.g., patient identifiers). In some implementations, the list of individual identifiers corresponds to the identifiers associated with a flag data attribute of 1. The randomization introduced by adding noise distributions to the flag data attributes can be adjusted to balance the utility of a machine learning model that processes the patient data and a risk of disclosure. For example, utility increases and risk of disclosure increases as the amount of noise in the patient data is increased. Similarly, utility decreases and risk of disclosure decreases as the amount of noise in the patient data approaches zero.

The training environment 502 receives a sample of the minimized data (i.e., a sample of the sequence of embedding vectors after being processed by one or more privacy enhancing techniques like dimensional reduction or noise insertion) from the health environment 504, a sample of randomized individual identifiers from the flag environment 506, and a sample of de-identified data from a de-identified dataset (e.g., data stored in the database 208 of the data environment 206 of FIG. 2).

The training environment 502 includes systems for training a machine learning model. The systems train the machine learning model according to the received model request and based on training data that includes the samples of randomized individual identifiers (i.e., individual identifiers associated with a FLAG data attribute of 1), embedding vectors, and de-identified data. The systems perform data processing steps including model tuning, model training, model output evaluation within the training environment 502. If a trained machine learning model generates output that meet pre-determined thresholds (e.g., utility threshold and/or disclosure threshold), the training environment 502 outputs a model object for use by downstream applications. In some implementations, the model object includes model deployment support files, evaluation metrics, and model configurations.

The consumer environment 510 receives the model object from the training environment 502. In addition, the consumer environment 510 receives the full set of minimized data (i.e., the full sequence of embedding vectors after being processed by one or more privacy enhancing techniques), the full set of de-identified patient data (e.g., access to the database 208 of FIG. 2), and the flag attributes as determined by the flag environment 506.

The consumer environment 510 applies the trained model received from the training environment 502 to the input data (e.g., the de-identified patient data). The flag attributes represent the outcome that the machine learning model is trained on. In other words, the machine learning model is trained to predict whether data corresponding to a particular individual is indicative of a particular set of criteria. The flag attribute was set by the flag environment to indicate (with some probability) that the individual corresponding to each flag attribute falls within the particular criteria. The consumer environment 510 receives the flag data to validate the machine learning model and the full set of de-identified patient data to process as an input to the machine learning model.

The consumer environment 510 includes a model scoring engine that assigns a score to each record in the de-identified patient data. The output of the machine learning model is a value indicative of an audience score that represents a likelihood that a particular record (associated with an individual) is associated with a target outcome (a member of a group of individuals that meet the criteria).

In some implementations, the machine learning model processes each record of the de-identified patient data and outputs a value indicative of likelihood that the respective record meets the criteria. In some implementations, each de-identified patient data record is categorized in a tiered group depending on the output value of the machine learning model. Each tiered group includes a range of percentages that are indicative of the likelihood that a record meets the criteria. In some implementations, thresholds are determined to place each de-identified patient data record in a corresponding tiered group. In some implementations, the consumer environment 510 outputs a list of patient identifiers (e.g., tokens or pseudonyms) and an associated probability that each patient meets the criteria or an associated tiered grouping of which the respective patient belongs. In some implementations, each tiered group represents groups of patients with similarly associated audience scores (e.g., a top 1%, top 10%, and so on).

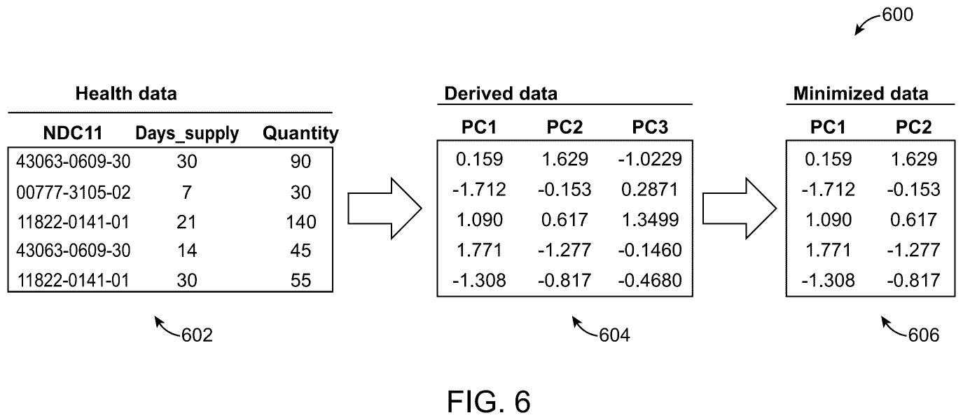

FIG. 6 illustrates an example data transformation 600. The example data transformation illustrates a transformation from de-identified patient data that includes health attributes to minimized data (e.g., a sequence of embedding vectors after a privacy enhancing technique is applied like dimensionality reduction). A health data table 602 shows a first column of data indicative of a unique identifier of a particular patient. The health data table 602 includes a second column of data indicative of a number of days supply associated with a particular drug. The health data table 602 includes a third column of data indicative of a quantity of the drug prescribed to the associated individual.

A derived data table 604 shows principal components (i.e., outputs of a PCA procedure) of the health data illustrated in the health data table 602, as described in relation to FIG. 2. In some implementations, the example columns of the derived data table 604 represent vectors along directions in an embedding space that exhibit the highest variance among all of the vectors in the embedding space. In some implementations, the principal components represented in the derived data table 604 are linear combination of a set of original features determined based on the data represented in the health data table 602.

A minimized data table 606 shows a reduced set of principal components (i.e., the principal components from the PCA procedure with “PC3” dropped). The dropping of one of the principal components is an example of a privacy enhancing technique. A reduction in the number of principal components that describes the health data is equivalent to adding noise to the health data and reduces a likelihood that the identities of patients represented by the rows of the data tables 602-604 are disclosed.

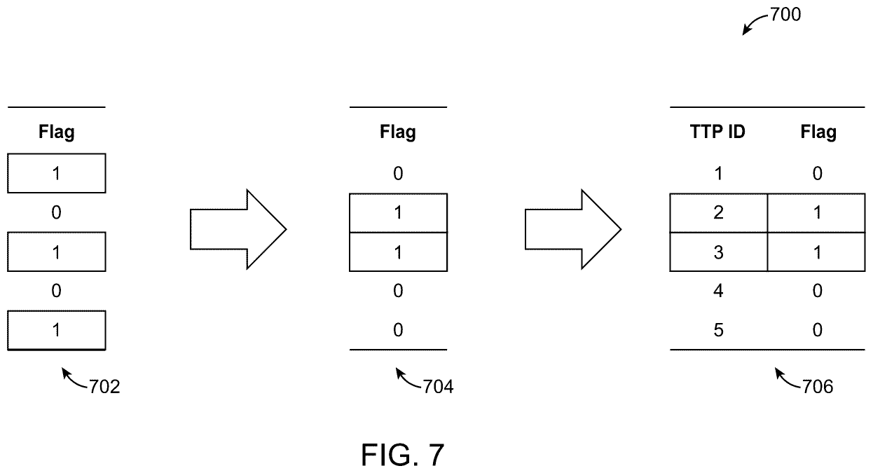

FIG. 7 illustrates an example randomization 700 of health data. As described in relation to the flag environment 506 described in FIG. 5, a system can retrieve data from a health data database based on a model request with an associated audience definition. For example, an audience definition is “patients aged 18 to 30 who have been diagnosed with Type II diabetes and are not currently on Metformin with a 5 year lookback.” In response to receiving a model request with an audience definition, the system can retrieve a sample of health data from a health data database and search the sample of health data for data entries that contain the criteria defined by the audience definition (e.g., 18-30 years old, Type II diabetes, and not on Metformin in the last 5 years).

The system generates a flag attribute for each entry of the sampled health data. A flag attribute of 0 indicates the entry does not meet the inclusion criteria. A flag attribute of 1 indicates the entry meets the inclusion criteria. A first sequence 702 of flag attributes illustrates five sample health data entries, in which the first, third, and fifth entries meet the inclusion criteria while the second and fourth entries do not meet the inclusion criteria.

The system performs a randomization process on the list of flag attributes such that with a pre-determined probability, the flag attributes are inverted. For example, the second sequence 704 of flag attributes illustrates the five sample health data entries after randomization. The result is the first, second, and fifth data entries are inverted. The randomization process introduces uncertainty into the flag attribute and results in noisy statistical outputs during analysis as a privacy enhancing technique.

A data structure 706 includes the randomized flags from the second sequence 704 and associated joined tokens that uniquely identify an individual/patient associated with the health data entry. The token is indicative of a particular individual and the flag value is a noisy indication of whether the particular individual meets the criteria of the audience definition. The token and flag attributes can be passed to a training environment (e.g., the training environment 502) for training a machine learning model.

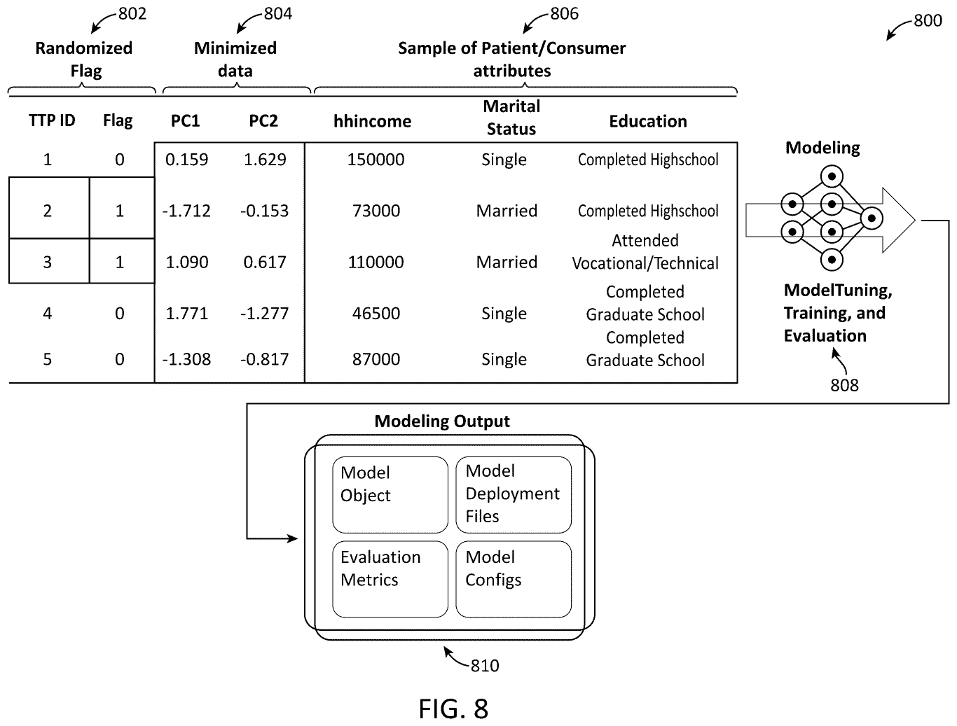

FIG. 8 illustrates an example dataset 800 that includes randomized flag attributes 802, a minimized dataset 804, and sample patient/consumer attribute data 806. The example dataset 800 is processed by a training environment (e.g., the training environment 502) and includes data generated in a flag environment (e.g., the flag environment 506) and a health environment (e.g., the health environment 504).

The randomized flag attributes 802 represent a list of identifiers (e.g., patient identifiers) with associated flag attributes that indicate (with some probability) that the associated health data meets criteria defined by an audience definition. For each row of the randomized flag attributes 802 (i.e., for each individual or patient), a value associated with the minimized dataset 804 is appended. Similarly, the sample patient and/or consumer attribute data 806 are appended for each individual that can include data attributes like household income, marital status, and education level of the individual.

The training environment can implement one or more modeling procedures 808. For example, the modeling procedures 808 can include training an initial machine learning model configured to learn relationships between features and target variables of interest (as defined by the audience definition). Furthermore, the modeling procedures 808 can include adjusting the model's parameters to minimize prediction errors (i.e., maximize a utility metric) and performing one or more cross-validation techniques to validate model performance.

Upon generating a machine learning model that meets pre-defined utility and risk of disclosure thresholds, the training environment can output a modeling output 810 that can include a model object (e.g., weight matrices or executable files), model deployment files, evaluation metrics, and model configuration data.

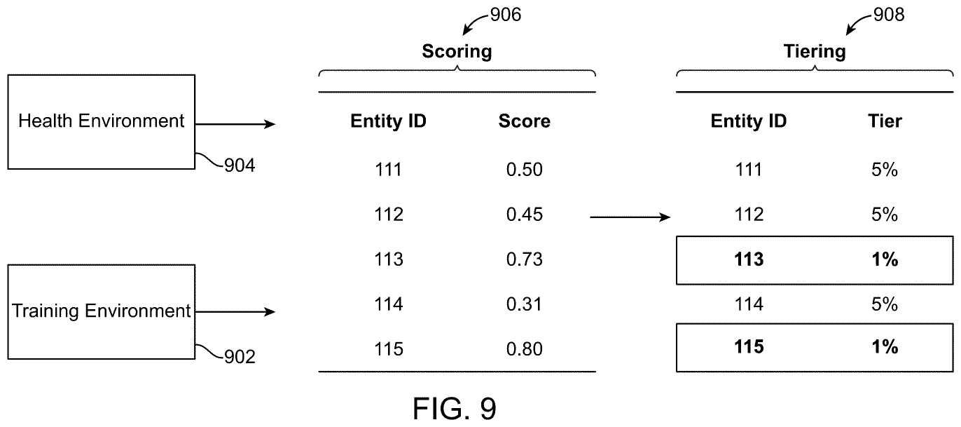

FIG. 9 illustrates an example evaluation process 900 of machine learning model outputs. Model outputs from a training environment 902 (e.g., the training environment 502) are received by a consumer environment (e.g., consumer environment 510). The model outputs include model files that enable an implementation of the trained machine learning model in the consumer environment. In addition, a health environment 904 (e.g., health environment 504) outputs are received by the consumer environment. The health environment outputs include minimized data (e.g., a compressed numerical representation of de-identified health data as a sequence of embedding vectors).

The consumer environment processes the minimized data from the health environment 904 with the received model output from the training environment 902. The model output includes all weights, configuration files, and other information required to implement the trained machine learning model generated in the training environment 902. The trained machine learning model processes the data received from the health environment 904 and generates an audience score. A scoring table 906 illustrates a first column of patient identifiers (entity identifiers), in which each row corresponds to a particular patient or entity. The scoring table 906 illustrates example audience scores (output values of the trained machine learning model) that are indicative of a likelihood that the corresponding individual represented by the patient identifier meets the particular criteria, as defined by a flag environment, and used to train the machine learning model in the training environment 902.

A tiering table 908 represents a tiered grouping representation of the data represented in the scoring table 906. One or more thresholds are set to define particular tiered groupings. For example, the tiering table 908 categorizes each patient identifier as in the top 5% or the top 1% in likelihood. The percentages in the “tier” column of the tiering table 908 represents groups of individuals with similar audience scores (represented in the “score” column of the scoring table 906). FIG. 9 illustrates an example evaluation process 900 of machine learning model outputs. Model outputs from a training environment 902 (e.g., the training environment 502) are received by a consumer environment (e.g., consumer environment 510). The model outputs include model files that enable an implementation of the trained machine learning model in the consumer environment. In addition, a health environment 904 (e.g., health environment 504) outputs are received by the consumer environment. The health environment outputs include minimized data (e.g., a compressed numerical representation of de-identified health data as a sequence of embedding vectors).

The consumer environment processes the minimized data from the health environment 904 with the received model output from the training environment 902. The model output includes all weights, configuration files, and other information required to implement the trained machine learning model generated in the training environment 902. The trained machine learning model processes the data received from the health environment 904 and generates an audience score. A scoring table 906 illustrates a first column of patient identifiers (entity identifiers), in which each row corresponds to a particular patient or entity. The scoring table 906 illustrates example audience scores (output values of the trained machine learning model) that are indicative of a likelihood that the corresponding individual represented by the patient identifier meets the particular criteria, as defined by a flag environment, and used to train the machine learning model in the training environment 902.

A tiering table 908 represents a tiered grouping representation of the data represented in the scoring table 906. One or more thresholds are set to define particular tiered groupings. For example, the tiering table 908 categorizes each patient identifier as in the top 5% or the top 1% in likelihood. The percentages in the “tier” column of the tiering table 908 represents groups of individuals with similar audience scores (represented in the “score” column of the scoring table 906).

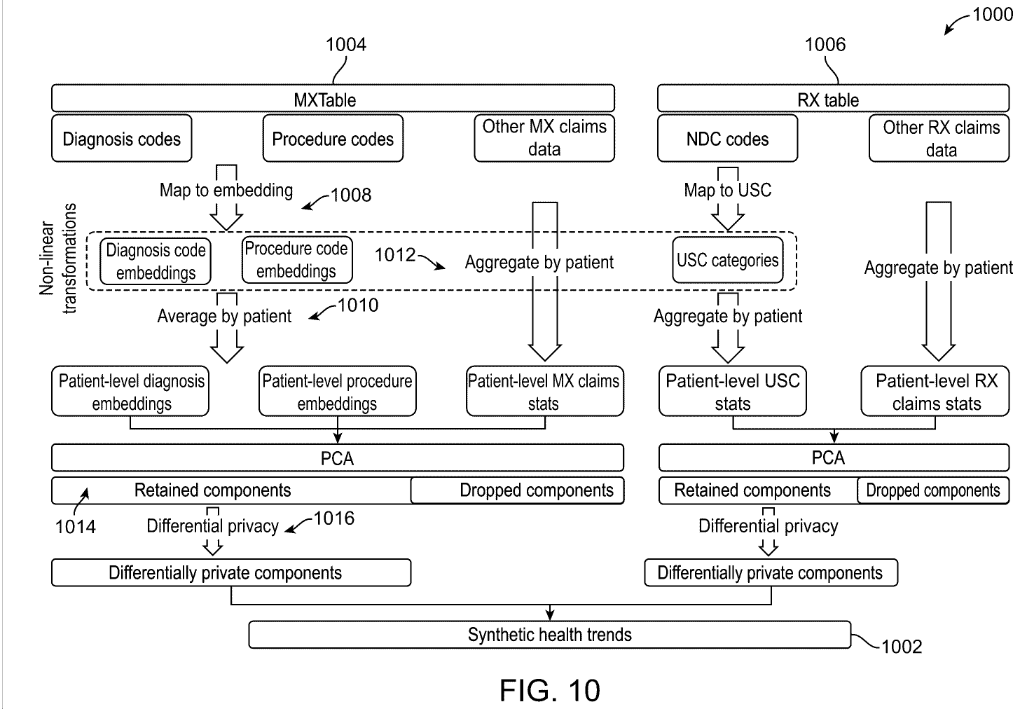

FIG. 10 illustrates an example process 1000 for generating synthetic health trends 1002. The process 1000 can be performed by a system similar to the system 200, which can include one or more computer systems. The process 1000 illustrates a combination of data from a first data environment 1004 that stores medical data and a second data environment 1006 that stores pharmacy-related data. The first data environment 1004 stores data that include diagnosis codes and procedure codes (i.e., ICD-10 codes, or other alphanumeric codes), as well as other medical claims data. Similarly, the second data environment 1006 stores data that include National Drug Codes (NDC codes) and other pharmacy-related claims data.

The process includes mapping the codes 1008 of the first data environment 1004 (e.g., the diagnosis codes and the procedure codes) to respective embeddings. For example, the diagnosis codes are mapped, via a nonlinear transformation as described in relation to FIG. 2, to diagnosis code embeddings. As another example, the procedure codes are mapped to procedure code embeddings. In some implementations, the process of converting codes to embeddings captures semantic relationships between codes. In some implementations, the system determines a top threshold number (e.g., top three) codes (e.g., diagnosis codes) per patient to reduce data dimensionality while preserving key trends in the data. In some implementations, additional filtering takes into account data recency and/or co-occurrence of codes to determine relevance of the codes.

The system maps the codes (e.g., ICD-10 codes) to embedded representations with a variety of techniques. For example, by aggregating repeated codes, the system can represent the repeated codes with pretrained embedding vectors. The embedding vectors (e.g., 1024-dimensional vectors) capture semantic relationships between diagnosis and procedures and can be further refined with torch autoencoders to reduce dimensionality without losing critical information. Similarly, the system can restructure procedure codes into hierarchical groupings to facilitate generation of interpretable, feature-rich groupings.

The system can implement embedding procedures such as ICD2Vec, which utilizes a finetuned GatorTron model to map ICD-10 codes into dense 1024-size embedding vectors. The approach captures an essence of nearly 25,000 codes with five characters or fewer, ensuring minimal loss of information when truncating longer codes. Alternatives techniques include averaging embeddings for patients with a few codes and concatenating embeddings and applying zero-padding as necessary to manage large datasets. In addition, the system can assign a unique random float vector of a predefined size (e.g., [1,5]) to each code. This approach entails representing each patient's set of codes (e.g., ICD-10 codes) with these random vectors.

In some implementations, the system pre-processes numerical data present in the other medical-related data to compute summary statistics including mean, median, variance, and interquartile range for continuous variables like amount paid, fill rates, or lab results. In some implementations, the other medical-related data includes counts and/or percentages for attributes like prescription fills or missed appointments to capture patterns of care. In some implementations, fields based on temporal indicators are generated as aggregated time-related metrics, such as a number of transactions in a given period, can be used to preserve longitudinal context for a dataset related to a particular patient.

In some implementations, in addition to generated embedded representation of codes, the system applies additional clustering techniques to the codes to group related codes and to reduce noise in the dataset. In addition, in some implementations, the system transforms numeric attributes into standardized scores and/or ranks to enhance comparability across different numerical scales. In some implementations, the system normalizes numerical attributes to a standard range (e.g., z-scores or min-max normalization) to ensure comparability. In some implementations, the system adjusts generated embeddings to have a constant magnitude across features.

The embedded representation of the procedure codes reduces feature space dimensionality while retaining critical information and an opportunity for unifying disparate coding systems. All procedure codes across each data environment of a particular federated data environment are embedded with a common embedding process to ensure that each set of codes is represented in the same embedding space. For example, the system can utilize a biomedical language model (e.g., BioSimCSE) to create effective embedded representations of biomedical text.

The process includes averaging 1010 the respective embeddings by patient to generate patient-level diagnosis embeddings and patient-level procedure embeddings respectively. The process includes aggregating 1012 the other medical claims data to generate a patient-level medical claims statistics dataset.

The system performs a PCA process 1014 (e.g., mapping the embedded representations to the principal components of the embedding space, as described in relation to FIG. 2), to generate a set of embedding vectors that include (i) retained components (e.g., principal components), and (ii) dropped components (e.g., components that do not exhibit large variations). The system performs differential privacy operations 1016 on the retained components to generate one or more differentially private components.

Similar to the process performed in relation to the first data environment 1004 related to medical data, the process includes data transformations of data stored in the second data environment 1006 in relation to the pharmacy-related data. The process maps the NDC codes to set of categories that belong to a uniform system of classification (USC). In some implementations, the mapping is performed by one or more non-linear transformations. The process includes aggregating the USC categories by patient to generate patient-level USC statistics. By mapping NDC codes with hierarchical USC codes, the system facilitates an efficient representation of information while hiding nonlinear relationships between data entries.

The NDC codes exhibit a flat data structure with minimal semantic depth. Each code indicates a specific product (e.g., a medication) but does not show relationships between similar products. In addition, use of NDC codes results in challenges in grouping related codes since identifying product connections requires manual effort or external knowledge, raising complexity and a risk of errors. Use of NDC codes also results in limited feature engineering opportunities with an absence of hierarchical structure.

Alternative to NDC codes, USC codes exhibit a hierarchical structure that support intuitive groupings, since codes are arranged in multi-level categories for better aggregation of similar products. In addition, USC codes lead to flexible categorization and enhanced feature engineering.

Similarly, the system aggregates the other pharmacy-related data by patient to generate patient-level pharmacy-related claims statistics. The system performs a PCA to generate retained components and dropped components. The system performs differential privacy on the retained components to generate a set of respective differentially private components.

The system combines the respective sets of differential private components from the first data environment 1004 and the second data environment 1006 to generate the synthetic health trends 1002.

In some implementations, the system performs one or more validation processes on the synthetic health trends 1002 to ensure the trends 1002 represent a balance of utility for both specific applications (e.g., audience modeling) and privacy protection. Some testing steps performed by the system include testing different parameter configurations for PCA (e.g., variance thresholds, number of principal components), against audience quality metrics like classification accuracy or clustering performance. In addition, the steps can include evaluating information retained in the embedded representations by calculating explained variance for the synthetic health trends 1002, ensuring sufficient fidelity for audience modeling. In addition, the steps can include assessing a distortion metric, such as reconstruction error or differential privacy loss, to measure a trade-off between privacy guarantees and data utility. In addition, the steps can include validating the synthetic health trends 1002 with a focus on preserving key relationships between fields while minimizing overfitting and/or information leakage.

In some implementations, the system determines variables in a dataset that are suitable for generating the synthetic health trends 1002 based on a number of missing values in the dataset. For example, the system can consider a percentage of missing values to be a threshold over which the data is excluded from the modeling process. In some cases, the system can include data with a large number of missing values if the sparsity of the data is expected (e.g., rare but important events).

In some implementation, the system performs operations related to ensuring a quality of final results such that the synthetic health trends 1002 meet defined benchmarks for accuracy, privacy, and usability in particular applications (e.g., audience modeling applications). The quality control steps can include audience modeling performance evaluation, assessing an effectiveness of the synthetic health trends 1002 by testing audience models on key tasks, such as cohort segmentation or classification, and measured performance metrics (e.g., precision, recall, and F1 score). Furthermore, the quality control steps can include comparing results against baseline models using raw or less-transformed data to verify improvements or acceptable trade-offs.

In some implementations, the system can perform operations related to validating privacy of the data represented in the synthetic health trends 1002. For example, the system can confirm that privacy thresholds, such as differential privacy epsilon values or distortion metrics, meet pre-specified tolerances. Similarly, the system can conduct adversarial tests to evaluate a risk of re-identification, ensuring compliance with established privacy guidelines.

In some implementations, the system performs operations related to checking an integrity of determined data relationships. For example, the system can test for a preservation of clinically relevant relationships in the data (e.g., correlations between health codes and outcomes) present in the synthetic health trends 1002.

In some implementations, the system can perform operations related to testing the robustness of the system and in particular, the synthetic health trends 1002. For example, the system can introduce variations to the input data (e.g., simulated noise and/or missing data) to test the resilience (e.g., reliability) of the synthetic health trends 1002. In addition, the system can validate the trends 1002 against multiple PCA configurations to identify the most stable and generalizable parameters settings for the principal components.

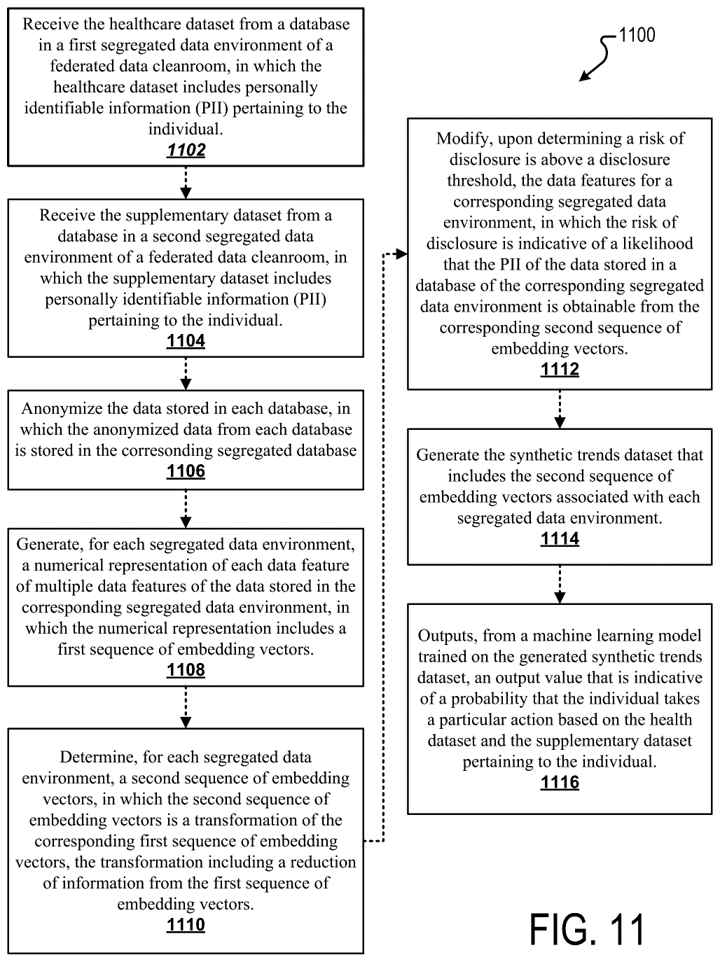

FIG. 11 is a flow diagram of an example process 1100 for generating a synthetic trends dataset by securely combining a healthcare dataset pertaining to an individual with a supplementary dataset pertaining to the individual. The process can be performed by a system similar to the system 200, which can include one or more computer systems.

The system receives (1102) the healthcare dataset from a database in a first segregated data environment of a federated data cleanroom. The healthcare dataset includes personally identifiable information (PII) pertaining to the individual. Furthermore, the system receives (1104) the supplementary dataset from a database in a second segregated data environment of the federated data cleanroom, in which the supplementary data includes PII pertaining to the individual as well. In some cases, the data from the first and second segregated data environments are restricted for direct comingling due to regulatory, privacy, intellectual property, or other restrictions. In some cases, the PII in the healthcare dataset and/or the supplementary dataset include data fields related to the individual that include name, social security number, address, among others. In some cases, the supplementary dataset is related to consumer activity of the individual, in which the supplementary dataset represents consumer data.

The system anonymizes (1106) the data stored in each database, in which the anonymized data from each database is stored in the corresponding segregated database. In some implementations, the system anonymizes the data by tokenizing PII, replacing sensitive information with pre-defined tokens, or other approaches to removing PII and/or otherwise de-identifying the data stored in each database.

The system generates (1108), for each segregated data environment, a numerical representation of each data feature of multiple data features of the data stored in the corresponding segregated data environment, in which the numerical representation includes a first sequence of embedding vectors. In some implementations, the sequence of embedding vectors is generated by a dimensionality reduction technique like PCA, or related techniques that map a dataset onto a lower dimensional embedding space.

The system determines (1110), for each segregated data environment, a second sequence of embedding vectors, in which the second sequence of embedding vectors is a transformation of the corresponding first sequence of embedding vectors, the transformation including a reduction of information from the first sequence of embedding vectors. In some implementations, the reduction of information includes further reducing the dimensionality of the first sequence of embedding vectors, introducing a lossy compression of the first sequence of embedding vectors, adding noise to the first sequence of embedding vectors (e.g., or multiple sources of noise)

The system modifies (1112), upon determining a risk of disclosure is above a disclosure threshold, the data features for a corresponding segregated data environment, in which the risk of disclosure is indicative of a likelihood that the PII of the data stored in a database of the corresponding segregated data environment is obtainable from the corresponding second sequence of embedding vectors. In some implementations, the modification of the data features includes modifying one or more parameters of the PCA procedure (e.g., a number of principal components).

The system generates (1114) the synthetic trends dataset that includes the second sequence of embedding vectors associated with each segregated data environment. In some implementations, the synthetic trends dataset includes transformed (e.g., with added noise) embedded representations of multiple datasets that originate from segregated data environments of a federated data cleanroom.

The system outputs (1116), from a machine learning model trained on the generated synthetic trends dataset, an output value that is indicative of a probability that the individual takes a particular action based on the health dataset and the supplementary dataset pertaining to the individual. For example, in the example case that the supplementary dataset is a consumer dataset (i.e., contains consumer-related data associated with the individual), the particular action can be a purchase of a particular healthcare-related product (e.g., drug, therapy, etc.). The combination of consumer data with healthcare data provides an end user richer insights into likely behavior of the individual without putting the PII of the individual at risk of disclosure.

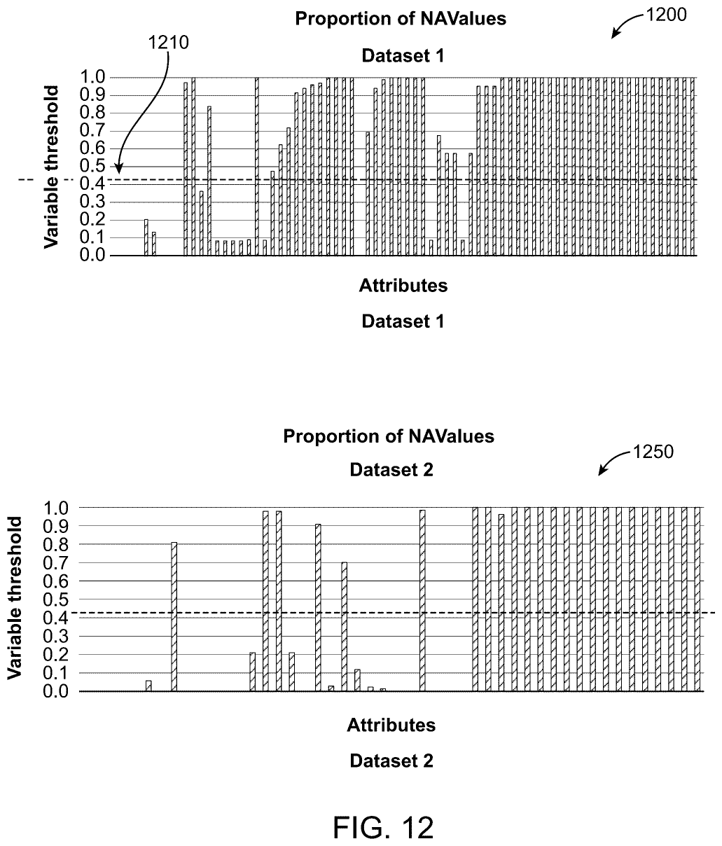

FIG. 12 is a graphical representation of a threshold pre-processing approach. The graphical representation depicts two plots. The two plots include a first plot 1200 associated with a first dataset that depicts a proportion of values for each attribute of the first dataset that have a value of “NA” (i.e., a missing value). The two plots include a second plot 1250 associated with a second dataset that depicts a proportion of values for each attribute of the second dataset that have a value of NA. The representation includes a threshold line 1210 overlayed on the first plot and the second plot, in which the threshold line 1210 represents a threshold proportion of NA values for a particular attribute, above which the particular attribute is not considered as a suitable attribute of the corresponding dataset and is thus ignored (i.e., remove from the corresponding dataset) when considered for modeling purposes.

In general, data attributes with high proportions of missing values (NA values) often provide minimal useful information for general modeling tasks, risking introducing noise and bias in a related analysis. In some implementations, data quality is improved by excluding high-NA data attributes of a dataset. In some implementations, the value of the threshold line 1210 depends on the particular type of modeling. For example, a general predictive modeling task has little use for high-NA data attributes. On the other hand, a modeling approach predicting rare events gains valuable (and necessary) insight from sporadic (i.e., high-NA) datasets.

In an example analysis represented in the graphical representation, a threshold line 1210 positioned at 50% (e.g., half of the values of a data attribute are missing) yields a reduction of data attributes of the first dataset from 74 to 22 data attributes and yields a reduction of data attributes of the second dataset from 48 to 24 data attributes. The reduced number of data attributes results in a smaller modeling space and a lower computational requirement for downstream data processing steps.

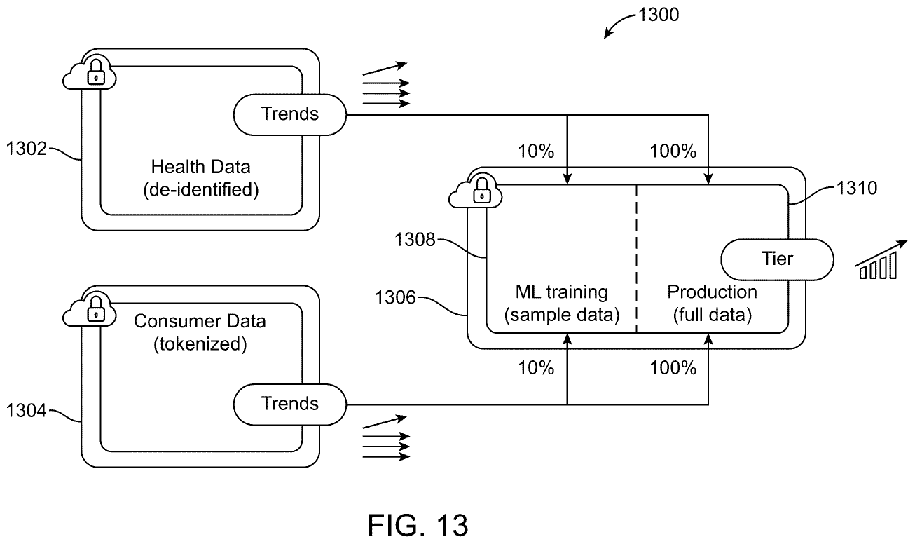

FIG. 13 illustrates an example system 1300 for combining data from two segregated data environments. The segregated data environments include a health data environment 1302 and a consumer data environment 1304. In some implementations, the health data environment 1302 includes one or more databases that store health-related data associated with clinical trials, medical procedures, medications, etc., for multiple patients. In some implementations, the consumer data environment 1304 includes one or more consumer-related data associated with consumer behavior of individuals. In some cases, a subset of the individuals (patients) associated with the data stored in the health data environment 1302 overlap with a subset of individuals associated with the data stored in the consumer data environment 1304.

In some implementations, consumer data stored in the consumer data environment 1304 is segregated from health date stored in the health data environment 1302 (e.g., to comply with relevant regulations). In some implementations, record keys of databases of the health data environment 1302 are stored in a tokenized form that is unique to a particular use case (e.g., unique to a particular implementation of the system 1300). In some applications, the data from the segregated environments 1302, 1304 must be combined to derive insights related to consumer activity and health data associated with particular individuals or cohorts. Before combining the data from the segregated environments 1302, 1304, as described in the present disclosure in relation to the descriptions of the preceding figures, data from each environment 1302, 1304 is first processed with embedding procedures (e.g., PCA) and transformed with one or more privacy-enhancing processes (e.g., noise injection).

The embedded representations of the data from each environment 1302, 1304 are output as synthetic trends before entering a shared modeling environment 1308. For example, a system of each respective environment 1302, 1304 (e.g., processors that execute instructions with access to data stored in respective segregated environments 1302, 1304) derives the synthetic trends from a differentially private embedding space (e.g., latent space of PCA) to minimize a risk of reconstructing the original data stored in the respective segregated environment.

In some implementations, the modeling environment 1308 includes a training environment 1308 and a production environment 1310. In some implementations, the training environment 1308 processes a subset (e.g. 10%) of the synthetic trend data for training purposes. In some implementations, the production environment 1310 implements a trained machine learning model and processes a full data set (e.g., 100%) associated with the combined synthetic datasets received from the heath data environment 1302 and the consumer data environment 1304.

In some implementations, model outputs from the production environment 1310 are converted to tiers that form audience data insights. For example, audience data insights can include “90th percentile matched to internet devices.”

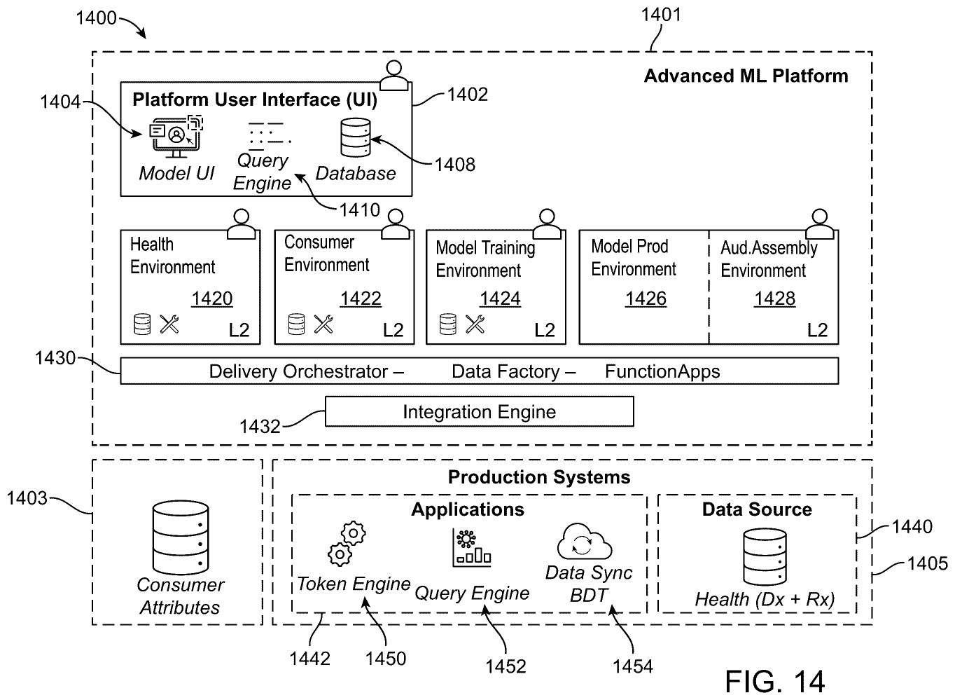

FIG. 14 illustrates an example system architecture 1400 that includes a machine learning (ML) platform 1401, a consumer data database 1403, and a health data environment 1405 that includes a health data database 1440 and production systems that include one or more applications 1442 (e.g., a token engine 1450, a query engine 1452, and a data sync engine 1454).

In some implementations, a user interacts with a platform user interface (UI) 1402 to manage one or more processes executed by servers of the system architecture 1400. For example, a model UI 1404, an interface to a query engine 1410, and a database interface 1408 to allow a user to query and view query results of one or more databases can allow a user to define cohorts of individuals, extract and review relevant health data, among other process management and data analysis actions.

The ML platform 1401 includes multiple segregated data environments. The segregated data environments include a health data environment 1420, a consumer data environment 1422, a model training environment 1424, and a production environment that includes a model production environment 1426 and an audience assembly environment 1428. In some cases, regulatory (and others) considerations prevent data from one data environment to be comingled with data from another environment. For example, in many cases, data from the health data environment 1420 should not be directly combined with data from the consumer data environment 1422.

In some implementations, each environment includes one or more data processors (e.g., processors, servers, etc., that can execute instructions associated with data processing tasks) and one or more databases. In addition, in some implementations, each environment can receive instructions from one or more shared resources 1430 (e.g., data delivery orchestration engine, data factories, and other function applications). Furthermore, in some implementations, the ML platform 1401 includes an integration engine 1432 that executes instructions for integrating data from various environments.

In some implementations, each processor is associated with executable instructions that perform tasks as described in the present disclosure (e.g., tokenization, embeddings generation, privacy enhancing techniques, etc.). In addition, in some implementations, each processor accesses one or more associated databases (e.g., consumer data, health data, etc.).

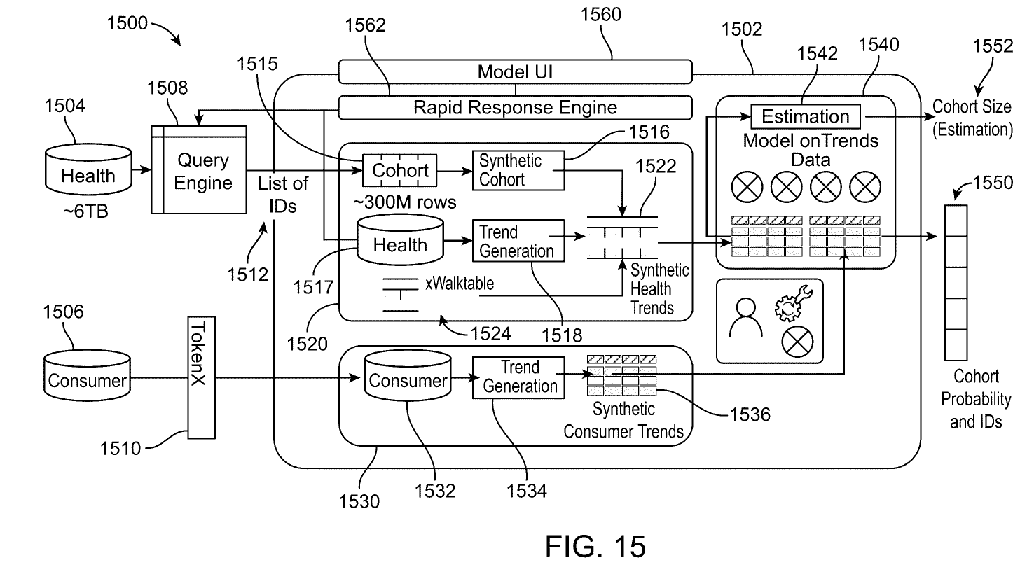

FIG. 15 is an example system 1500 that includes one or more databases, one or more servers, and associated instructions for processing data from two segregated databases and generating an output of a machine learning model that processes synthetic trend data originating from each of the segregated databases. The example system 1500 includes a health data database 1504 (e.g., health data records that can be upwards of several TB of data) and a consumer data database 1506. In some cases, due to regulatory and other constraints (contractual, intellectual property, etc.) cannot be directly combined and analyzed jointly.

The system 1500 includes a model user interface (model UI 1560), in which a user can initiate an analytics job that requires a combination of data from the health data database 1504 and the consumer data database 1506. The model UI 1560 is communicatively coupled with a rapid response engine 1562 that receives queries from the model UI 1560 and executes instructions associated with the request.

For example, the rapid response engine 1562 can initiate one or more data queries to the health data database 1504 via a query engine 1508 that accesses data stored in the health data database 1504. The query engine 1508 queries the health data database 1504 for a subset of records that satisfy a particular criteria and transmits a list of IDs 1512 to a health data environment 1520. The health data environment 1520 receives the list of IDs 1512 as a patient cohort 1515. In some implementations, the patient cohort 1515 includes data related to a subset of patients (e.g., 300M rows) of the health data database 1504.

In some implementations, the health data environment 1520 processes the patient cohort 1515 and generates a synthetic patient cohort 1516. The synthetic patient cohort 1516 is an embedded representation of the patient cohort 1516 (e.g., a vector or numerical representation of the patient cohort 1516). The health data environment 1520 includes a subset of data from the health data database 1504 stored in a health data subset database 1517, which stores health data associated with the patients that belong to the patient cohort 1515. The health data environment 1520 generates synthetic trends 1518, as described in relation to the descriptions of the preceding figures of this disclosure (e.g., PCA, noise injection, etc.). The health data environment 1520 combines the synthetic cohort 1516 with the synthetic trends 1518 to generate the synthetic health trends 1522. In some implementation, the health data environment 1520 matches data represented in the synthetic cohort 1516 with data represented in the synthetic trends 1518 that correspond to the same patient via a cross walk table 1524 that stores a mapping of relationships between attributes in the data.

The system 1500 includes a consumer data environment 1530 that receives tokenized consumer data from the consumer database 1506. In some implementations, a tokenization engine 1510 tokenizes the consumer data before it is received by the consumer data environment 1530. The consumer data environment 1530 accesses a subset of consumer data from a consumer data subset database 1532. The consumer data environment 1530 transforms the consumer data stored in the consumer data subset database 1532 into an embedded representation (e.g., via PCA with noise injection or differential privacy) by a trends generation 1534 engine to generate a dataset of synthetic consumer trends 1536.

The synthetic consumer trends 1536 and the synthetic health trends 1522 (embedded representation of the respective original data) are received by a model environment 1540. The model environment includes one or more estimators 1542 and can execute data processing tasks (e.g., combining the trends 1522 and 1536 into a single dataset). The model environment 1540 can output an estimation of the cohort size 1552 via the one or more estimators 1542 and a list of cohort probability IDs, in which the probability is related to the particular request received from a user by the model UI 1560.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory program carrier for execution by, or to control the operation of, data processing apparatus. Alternatively, or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. The computer storage medium can be a machine-readable storage device, a machine-readable storage substrate, a random or serial access memory device, or a combination of one or more of them. The computer storage medium is not, however, a propagated signal.

The term “data processing apparatus” encompasses all kinds of apparatus, devices, and machines for processing data, including by way of example a programmable processor, a computer, or multiple processors or computers. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, a software module, a script, or code) can be written in any form of programming language, including compiled or interpreted languages, or declarative or procedural languages, and it can be deployed in any form, including as a stand-alone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

Computers suitable for the execution of a computer program include, by way of example, can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet.

While this specification contains specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

In addition to the embodiments described above, the following embodiments are also innovative:

Embodiment 1 is a method for generating a synthetic trends dataset by securely combining a healthcare dataset pertaining to an individual with a supplementary dataset pertaining to the individual, the method comprising:

receiving the healthcare dataset from a database in a first segregated data environment of a federated data cleanroom, the healthcare dataset comprising personally identifiable information (PII) pertaining to the individual,
receiving the supplementary dataset from a database in a second segregated data environment of the federated data cleanroom, the supplementary data comprising PII pertaining to the individual,
anonymizing the data stored in each database, wherein the anonymized data from each database is stored in the corresponding segregated database,
generating, for each segregated data environment, a numerical representation of each data feature of a plurality of data features of the anonymized data stored in the corresponding segregated data environment, wherein the numerical representation comprises a first sequence of embedding vectors,
determining, for each segregated data environment, a second sequence of embedding vectors, wherein the second sequence of embedding vectors is a transformation of the corresponding first sequence of embedding vectors, the transformation comprising a reduction of information from the first sequence of embedding vectors,
modifying, upon determining a risk of disclosure is above a disclosure threshold, the plurality of data features for a corresponding segregated data environment, wherein the risk of disclosure is indicative of a likelihood that the PII of the data stored in a database of the corresponding segregated data environment is obtainable from the corresponding second sequence of embedding vectors,
generating the synthetic trends dataset that comprises the second sequence of embedding vectors associated with each segregated data environment, and
outputting, from a machine learning model trained on the generated synthetic trends dataset, an output value that is indicative of a probability that the individual takes a particular action based on the health dataset and the supplementary dataset pertaining to the individual.

Embodiment 2 is the method of embodiment 1, wherein the transformation of the first sequence of embedding vectors comprises reducing a dimensionality of the first sequence of embedding vectors.

Embodiment 3 is the method of any of embodiments 1-2, wherein the transformation of the first sequence of embedding vectors comprises a lossy compression of the first sequence of embedding vectors.

Embodiment 4 is the method of any of embodiments 1-3, wherein the transformation of the first sequence of embedding vectors comprises adding noise to the first sequence of embedding vectors, wherein the noise comprises one or more sources of noise.

Embodiment 5 is the method of any of embodiments 1-4, wherein the generation of the first sequence of embedding vectors for each segregated data environment comprises a principal component analysis of the corresponding dataset.

Embodiment 6 is the method of any of embodiments 1-5, further comprising generating a token from the PII of each dataset pertaining to the individual, wherein the token is operative to link the corresponding dataset to data stored outside of the federated data cleanroom.

Embodiment 7 is the method of any of embodiments 1-6, further comprising:
determining a utility of the dataset, wherein the utility is indicative of a quality of the dataset with respect to a particular task,
determining that the utility of the dataset is below a utility threshold that represents a minimum required quality of insights generated based on analytics of the dataset,
modifying, based on the utility of the dataset being below the utility threshold, one or more of the determined data features to increase the utility of the dataset, and
after the modifying, outputting insights generated based on analytics of the dataset.

Embodiment 8 is the method of any of embodiments 1-7, wherein determining the risk of disclosure comprises determining a k-anonymity metric, wherein the k-anonymity metric depends on a signal-to-noise ratio (SNR) and a similarity probability of each data point of the second sequence of embedding vectors.

Embodiment 9 is the method of any of embodiments 1-8, wherein generating the first sequence of embedding vectors comprises capturing a variance of the anonymized data in fewer dimensions than the dimensionality of the anonymized data.

Embodiment 10 is the method of any of embodiments 1-9, wherein the healthcare dataset comprises a plurality of alphanumeric codes, wherein each alphanumeric code is mapped to an embedding vector.

Embodiment 11 is a system comprising one or more computers, and one or more computer-readable media storing instructions that are operable, when executed by the one or more computers, to perform operations for generating a synthetic trends dataset by securely combining a healthcare dataset pertaining to an individual with a supplementary dataset pertaining to the individual, the operations comprising:
receiving the healthcare dataset from a database in a first segregated data environment of a federated data cleanroom, the healthcare dataset comprising personally identifiable information (PII) pertaining to the individual,
receiving the supplementary dataset from a database in a second segregated data environment of the federated data cleanroom, the supplementary data comprising PII pertaining to the individual,
anonymizing the data stored in each database, wherein the anonymized data from each database is stored in the corresponding segregated database,
generating, for each segregated data environment, a numerical representation of each data feature of a plurality of data features of the anonymized data stored in the corresponding segregated data environment, wherein the numerical representation comprises a first sequence of embedding vectors,
determining, for each segregated data environment, a second sequence of embedding vectors, wherein the second sequence of embedding vectors is a transformation of the corresponding first sequence of embedding vectors, the transformation comprising a reduction of information from the first sequence of embedding vectors,
modifying, upon determining a risk of disclosure is above a disclosure threshold, the plurality of data features for a corresponding segregated data environment, wherein the risk of disclosure is indicative of a likelihood that the PII of the data stored in a database of the corresponding segregated data environment is obtainable from the corresponding second sequence of embedding vectors,
generating the synthetic trends dataset that comprises the second sequence of embedding vectors associated with each segregated data environment, and
outputting, from a machine learning model trained on the generated synthetic trends dataset, an output value that is indicative of a probability that the individual takes a particular action based on the health dataset and the supplementary dataset pertaining to the individual.

Embodiment 12 is the system of embodiment 11, wherein the transformation of the first sequence of embedding vectors comprises one or more of reducing a dimensionality of the first sequence of embedding vectors, a lossy compression of the first sequence of embedding vectors, and adding noise to the first sequence of embedding vectors, wherein the noise comprises one or more sources of noise.

Embodiment 13 is the system of any of embodiments 11-12, wherein the generation of the first sequence of embedding vectors for each segregated data environment comprises a principal component analysis of the corresponding dataset.

Embodiment 14 is the system of any of embodiments 11-13, wherein the operations further comprise generating a token from the PII of each dataset pertaining to the individual, wherein the token is operative to link the corresponding dataset to data stored outside of the federated data cleanroom.

Embodiment 15 is the system of any of embodiments 11-14, the operations further comprising:
determining a utility of the dataset, wherein the utility is indicative of a quality of the dataset with respect to a particular task,
determining that the utility of the dataset is below a utility threshold that represents a minimum required quality of insights generated based on analytics of the dataset,
modifying, based on the utility of the dataset being below the utility threshold, one or more of the determined data features to increase the utility of the dataset, and
after the modifying, outputting insights generated based on analytics of the dataset.

Embodiment 16 is the system of any of embodiments 11-15, wherein determining the risk of disclosure comprises determining a k-anonymity metric, wherein the k-anonymity metric depends on a signal-to-noise ratio (SNR) and a similarity probability of each data point of the second sequence of embedding vectors.

Embodiment 17 is the system of any of embodiments 11-16, wherein generating the first sequence of embedding vectors comprises capturing a variance of the anonymized data in fewer dimensions than the dimensionality of the anonymized data.

Embodiment 18 is the system of any of embodiments 11-17, wherein the healthcare dataset comprises a plurality of alphanumeric codes, wherein each alphanumeric code is mapped to an embedding vector.

Embodiment 19 is a non-transitory computer-readable medium storing one or more instructions executable by a computer system to perform operations for generating a synthetic trends dataset by securely combining a healthcare dataset pertaining to an individual with a supplementary dataset pertaining to the individual, the operations comprising:
receiving the healthcare dataset from a database in a first segregated data environment of a federated data cleanroom, the healthcare dataset comprising personally identifiable information (PII) pertaining to the individual,
receiving the supplementary dataset from a database in a second segregated data environment of the federated data cleanroom, the supplementary data comprising PII pertaining to the individual,
anonymizing the data stored in each database, wherein the anonymized data from each database is stored in the corresponding segregated database,
generating, for each segregated data environment, a numerical representation of each data feature of a plurality of data features of the anonymized data stored in the corresponding segregated data environment, wherein the numerical representation comprises a first sequence of embedding vectors,
determining, for each segregated data environment, a second sequence of embedding vectors, wherein the second sequence of embedding vectors is a transformation of the corresponding first sequence of embedding vectors, the transformation comprising a reduction of information from the first sequence of embedding vectors,
modifying, upon determining a risk of disclosure is above a disclosure threshold, the plurality of data features for a corresponding segregated data environment, wherein the risk of disclosure is indicative of a likelihood that the PII of the data stored in a database of the corresponding segregated data environment is obtainable from the corresponding second sequence of embedding vectors,
generating the synthetic trends dataset that comprises the second sequence of embedding vectors associated with each segregated data environment, and
outputting, from a machine learning model trained on the generated synthetic trends dataset, an output value that is indicative of a probability that the individual takes a particular action based on the health dataset and the supplementary dataset pertaining to the individual.

Embodiment 20 is the medium of embodiment 19, wherein the transformation of the first sequence of embedding vectors comprises one or more of reducing a dimensionality of the first sequence of embedding vectors, a lossy compression of the first sequence of embedding vectors, and adding noise to the first sequence of embedding vectors, wherein the noise comprises one or more sources of noise.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A method for generating a synthetic trends dataset by securely combining a healthcare dataset pertaining to an individual with a supplementary dataset pertaining to the individual, the method comprising:
receiving the healthcare dataset from a database in a first segregated data environment of a federated data cleanroom, the healthcare dataset comprising personally identifiable information (PII) pertaining to the individual;
receiving the supplementary dataset from a database in a second segregated data environment of the federated data cleanroom, the supplementary data comprising PII pertaining to the individual;
anonymizing the data stored in each database, wherein the anonymized data from each database is stored in the corresponding segregated database;
generating, for each segregated data environment, a numerical representation of each data feature of a plurality of data features of the anonymized data stored in the corresponding segregated data environment, wherein the numerical representation comprises a first sequence of embedding vectors;
determining, for each segregated data environment, a second sequence of embedding vectors, wherein the second sequence of embedding vectors is a transformation of the corresponding first sequence of embedding vectors, the transformation comprising a reduction of information from the first sequence of embedding vectors;
modifying, upon determining a risk of disclosure is above a disclosure threshold, the plurality of data features for a corresponding segregated data environment, wherein the risk of disclosure is indicative of a likelihood that the PII of the data stored in a database of the corresponding segregated data environment is obtainable from the corresponding second sequence of embedding vectors;
generating the synthetic trends dataset that comprises the second sequence of embedding vectors associated with each segregated data environment; and
outputting, from a machine learning model trained on the generated synthetic trends dataset, an output value that is indicative of a probability that the individual takes a particular action based on the health dataset and the supplementary dataset pertaining to the individual.

2. The method of claim 1, wherein the transformation of the first sequence of embedding vectors comprises reducing a dimensionality of the first sequence of embedding vectors.

3. The method of claim 1, wherein the transformation of the first sequence of embedding vectors comprises a lossy compression of the first sequence of embedding vectors.

4. The method of claim 1, wherein the transformation of the first sequence of embedding vectors comprises adding noise to the first sequence of embedding vectors, wherein the noise comprises one or more sources of noise.

5. The method of claim 1, wherein the generation of the first sequence of embedding vectors for each segregated data environment comprises a principal component analysis of the corresponding dataset.

6. The method of claim 1, further comprising generating a token from the PII of each dataset pertaining to the individual, wherein the token is operative to link the corresponding dataset to data stored outside of the federated data cleanroom.

7. The method of claim 1, further comprising:
determining a utility of the dataset, wherein the utility is indicative of a quality of the dataset with respect to a particular task;
determining that the utility of the dataset is below a utility threshold that represents a minimum required quality of insights generated based on analytics of the dataset;
modifying, based on the utility of the dataset being below the utility threshold, one or more of the determined data features to increase the utility of the dataset; and
after the modifying, outputting insights generated based on analytics of the dataset.

8. The method of claim 1, wherein determining the risk of disclosure comprises determining a k-anonymity metric, wherein the k-anonymity metric depends on a signal-to-noise ratio (SNR) and a similarity probability of each data point of the second sequence of embedding vectors.

9. The method of claim 1, wherein generating the first sequence of embedding vectors comprises capturing a variance of the anonymized data in fewer dimensions than the dimensionality of the anonymized data.

10. The method of claim 1, wherein the healthcare dataset comprises a plurality of alphanumeric codes, wherein each alphanumeric code is mapped to an embedding vector.

11. A system comprising:
one or more computers;
one or more computer-readable media storing instructions that are operable, when executed by the one or more computers, to perform operations for generating a synthetic trends dataset by securely combining a healthcare dataset pertaining to an individual with a supplementary dataset pertaining to the individual, the operations comprising:
receiving the healthcare dataset from a database in a first segregated data environment of a federated data cleanroom, the healthcare dataset comprising personally identifiable information (PII) pertaining to the individual;
receiving the supplementary dataset from a database in a second segregated data environment of the federated data cleanroom, the supplementary data comprising PII pertaining to the individual;
anonymizing the data stored in each database, wherein the anonymized data from each database is stored in the corresponding segregated database;
generating, for each segregated data environment, a numerical representation of each data feature of a plurality of data features of the anonymized data stored in the corresponding segregated data environment, wherein the numerical representation comprises a first sequence of embedding vectors;
determining, for each segregated data environment, a second sequence of embedding vectors, wherein the second sequence of embedding vectors is a transformation of the corresponding first sequence of embedding vectors, the transformation comprising a reduction of information from the first sequence of embedding vectors;
modifying, upon determining a risk of disclosure is above a disclosure threshold, the plurality of data features for a corresponding segregated data environment, wherein the risk of disclosure is indicative of a likelihood that the PII of the data stored in a database of the corresponding segregated data environment is obtainable from the corresponding second sequence of embedding vectors;
generating the synthetic trends dataset that comprises the second sequence of embedding vectors associated with each segregated data environment; and
outputting, from a machine learning model trained on the generated synthetic trends dataset, an output value that is indicative of a probability that the individual takes a particular action based on the health dataset and the supplementary dataset pertaining to the individual.

12. The system of claim 11, wherein the transformation of the first sequence of embedding vectors comprises one or more of reducing a dimensionality of the first sequence of embedding vectors, a lossy compression of the first sequence of embedding vectors, and adding noise to the first sequence of embedding vectors, wherein the noise comprises one or more sources of noise.

13. The system of claim 11, wherein the generation of the first sequence of embedding vectors for each segregated data environment comprises a principal component analysis of the corresponding dataset.

14. The system of claim 11, wherein the operations further comprise generating a token from the PII of each dataset pertaining to the individual, wherein the token is operative to link the corresponding dataset to data stored outside of the federated data cleanroom.

15. The system of claim 11, the operations further comprising:
determining a utility of the dataset, wherein the utility is indicative of a quality of the dataset with respect to a particular task;
determining that the utility of the dataset is below a utility threshold that represents a minimum required quality of insights generated based on analytics of the dataset;
modifying, based on the utility of the dataset being below the utility threshold, one or more of the determined data features to increase the utility of the dataset; and
after the modifying, outputting insights generated based on analytics of the dataset.

16. The system of claim 11, wherein determining the risk of disclosure comprises determining a k-anonymity metric, wherein the k-anonymity metric depends on a signal-to-noise ratio (SNR) and a similarity probability of each data point of the second sequence of embedding vectors.

17. The system of claim 11, wherein generating the first sequence of embedding vectors comprises capturing a variance of the anonymized data in fewer dimensions than the dimensionality of the anonymized data.

18. The system of claim 11, wherein the healthcare dataset comprises a plurality of alphanumeric codes, wherein each alphanumeric code is mapped to an embedding vector.

19. A non-transitory computer-readable medium storing one or more instructions executable by a computer system to perform operations for generating a synthetic trends dataset by securely combining a healthcare dataset pertaining to an individual with a supplementary dataset pertaining to the individual, the operations comprising:
receiving the healthcare dataset from a database in a first segregated data environment of a federated data cleanroom, the healthcare dataset comprising personally identifiable information (PII) pertaining to the individual;
receiving the supplementary dataset from a database in a second segregated data environment of the federated data cleanroom, the supplementary data comprising PII pertaining to the individual;
anonymizing the data stored in each database, wherein the anonymized data from each database is stored in the corresponding segregated database;
generating, for each segregated data environment, a numerical representation of each data feature of a plurality of data features of the anonymized data stored in the corresponding segregated data environment, wherein the numerical representation comprises a first sequence of embedding vectors;
determining, for each segregated data environment, a second sequence of embedding vectors, wherein the second sequence of embedding vectors is a transformation of the corresponding first sequence of embedding vectors, the transformation comprising a reduction of information from the first sequence of embedding vectors;
modifying, upon determining a risk of disclosure is above a disclosure threshold, the plurality of data features for a corresponding segregated data environment, wherein the risk of disclosure is indicative of a likelihood that the PII of the data stored in a database of the corresponding segregated data environment is obtainable from the corresponding second sequence of embedding vectors;
generating the synthetic trends dataset that comprises the second sequence of embedding vectors associated with each segregated data environment; and
outputting, from a machine learning model trained on the generated synthetic trends dataset, an output value that is indicative of a probability that the individual takes a particular action based on the health dataset and the supplementary dataset pertaining to the individual.

20. The medium of claim 19, wherein the transformation of the first sequence of embedding vectors comprises one or more of reducing a dimensionality of the first sequence of embedding vectors, a lossy compression of the first sequence of embedding vectors, and adding noise to the first sequence of embedding vectors, wherein the noise comprises one or more sources of noise.
